(19) United States
(12) Patent Application Publication
(10) Pub. No.: US 2018/0161185 A1
(43) Pub. Date: Jun. 14, 2018

(54) ELECTROSPUN STENTS, FLOW DIVERTERS, AND OCCLUSION DEVICES AND METHODS OF MAKING THE SAME

(71) Applicants: eLum Technologies, Inc., Fremont, CA (US); Nanofiber Solutions, Inc., Hilliard, OH (US)

(72) Inventors: McKenzie Jacob KRESSLEIN, Columbus, OH (US); Brandon Javier BORJA, Edison, NJ (US); Jed JOHNSON, London, OH (US); Tyler Matthew GROEHL, Columbus, OH (US); Devan OHST, Columbus, OH (US); Quang Quoc TRAN, Atherton, CA (US); Kevin J. NELSON, Columbus, OH (US)

(21) Appl. No.: 15/842,228

(22) Filed: Dec. 14, 2017

Related U.S. Application Data
(60) Provisional application No. 62/434,258, filed on Dec. 14, 2016.
(60) Provisional application No. 62/487,138, filed on Apr. 19, 2017.

(51) Int. Cl. A61F 2/90; A61L 31/14; D01D 5/00

(52) U.S. Cl. CPC A61F 2/90; A61F 2/06; D01D 5/003; A61L 31/146

(57) ABSTRACT

The instant disclosure is directed to medical devices having a lattice framework. The lattice framework may comprise a plurality of interconnected polymeric electrospun fiber members, or may comprise one or more wires formed into a plurality of interconnected members. The lattice framework may be substantially tubular, or may have a bowtie or cone configuration. The medical devices described herein may find particular uses as stents, flow diverters, and occlusive devices. The instant disclosure is also directed to methods of making such medical devices, using electrospinning and processing techniques.

### CROSS-REFERENCE TO RELATED APPLICATIONS

This application claims priority to and benefit of U.S. Provisional Application Ser. No. 62/434,258, filed Dec. 14, 2016, entitled “Electrospun Stents and Flow Diverters and Methods of Making the Same,” and U.S. Provisional Application Ser. No. 62/487,138, filed Apr. 19, 2017, entitled “Electrospun Stents and Flow Diverters and Methods of Making the Same,” each of which is hereby incorporated herein by reference in its entirety.

### BACKGROUND

Peripheral endovascular stents are commonly used to combat coronary and peripheral artery diseases. These diseases can cause the narrowing of a blood vessel due to plaque buildup from fat or cholesterol deposition, known as atherosclerosis. This narrowing can lead to heart attacks, strokes, and other potentially fatal conditions if left untreated. Angioplasty procedures commonly involve breaking up plaque using a balloon, and placing a stent in the treated area to allow adequate blood flow and support the opened vessel as it heals.

Permanent stents may induce restenosis, or re-narrowing, of the vessel, to some degree due to compliance mismatch of the structure and inflammation at the interface. In addition, permanent stents may cause problems with endothelialization, increasing thrombosis rates. It is thought that stents are only needed temporarily, long enough for the vessel to heal, since late restenosis is associated with the permanent presence of the stent. Furthermore, currently available permanent stents may induce neointimal hyperplasia, i.e., thickening of vessel walls.

Current resorbable stents have been shown to increase thrombosis rates as compared to permanent stents. Thus, in order avoid restenosis, neointimal hyperplasia, repeated treatments, and other issues associated with permanent and currently available resorbable stents, there is a need to improve stents to reduce and/or eliminate these undesired outcomes.

Flow diverters are comparable to stents in their design and vascular applications. Cardiovascular diseases such as high blood pressure and atherosclerosis can lead to the weakening and thinning of arterial walls. An aneurysm is the ballooning of a weakened vessel, which can rupture if left untreated, releasing blood into surrounding tissue. Ruptured aneurysms may result in serious conditions, and even death.

Currently there are three methods of treating aneurysms, including surgical clipping, embolization, and flow diversion. Clipping is a high-risk procedure involving the removal of tissue or bone to access the aneurysm in order to clip it at its base stopping blood from entering the site. Embolization is an endovascular technique which utilizes a microcatheter to deploy permanent coils into the aneurysm in order to fill the ballooning aneurysm to displace the flow of blood. Although this procedure is less invasive than clipping, it poses the risk of rupturing the aneurysm during surgery, as well as recurrence due to post-operation recanalization. Stent-assisted coil embolization has increased the success rate of coiling. By navigating the microcatheter through the pores of a stent placed across the width of the aneurysm, coils are packed tighter and are prevented from being released into the vessel due to the support of the stent wall. Flow diversion employs a stent-like design with reduced porosity, which creates impedance. The reduction of blood flow into the aneurysm causes a pressure imbalance within the ballooning aneurysm, inducing an inflammatory response followed by the healing of the aneurysm.

The main drawback of currently available flow diverters is that they do not completely mechanically exclude the aneurysm from blood flow, but rather rely on the pressure differential created to initiate the remodeling of the vessel wall and ultimately seal the aneurysm. This process takes time and will not produce immediate results, thus leaving time for the aneurysm to progress. Therefore, a need exists to provide a flow diverter that alleviates one or more of the drawbacks associated with current flow diverter designs.

### SUMMARY

The instant disclosure is directed to medical devices, such as stents, flow diverters, occlusion and intrasaccular devices. In one embodiment, the medical device includes a substantially tubular lattice framework comprising a plurality of interconnected polymeric electrospun fiber members. In another embodiment, the medical device includes a substantially tubular lattice framework comprising at least one wire strand formed into a plurality of interconnected members. The medical device has an operational configuration having an operational diameter and operational length, and a delivery configuration having a delivery diameter and delivery length. Further, the operational diameter of the medical device is greater than the delivery diameter, and the operational length is less than or equal to the delivery length.

In another embodiment, the instant disclosure features a stent device. In one embodiment, the stent device includes a substantially tubular lattice framework comprising a plurality of interconnected polymeric electrospun fiber members. In another embodiment, the stent device includes a substantially tubular lattice framework comprising at least one wire strand formed into a plurality of interconnected members. The stent device has an operational configuration having an operational diameter and operational length, and a delivery configuration having a delivery diameter and delivery length. Further, the operational diameter of the medical device is greater than the delivery diameter, and the operational length is less than or equal to the delivery length.

In yet another embodiment, the instant disclosure features a flow diverter. In one embodiment, the flow diverter includes a substantially tubular lattice framework comprising a plurality of interconnected polymeric electrospun fiber members, and a polymeric electrospun mesh contacting at least two of the interconnected polymeric electrospun fiber members. In another embodiment, the substantially tubular lattice framework of the flow diverter comprises at least one wire strand formed into a plurality of interconnected members. The flow diverter has an operational configuration having an operational diameter and operational length, and a delivery configuration having a delivery diameter and delivery length. Further, the operational diameter of the medical device is greater than the delivery diameter, and the operational length is less than or equal to the delivery length. In some embodiments, the tubular lattice framework of the flow diverter includes a metal core.

In still another embodiment, a medical device may include a lattice framework comprising at least one strand formed into a plurality of interconnected members, and a polymeric electrospun mesh contacting at least two of the interconnected members and having a pore size. The lattice framework of such a medical device may have, in some embodiments, a substantially tubular shape, a bowtie shape, a cone shape, or a combination thereof. In certain embodiments, the polymeric electrospun mesh may extend over one or both ends of the medical device. Such a medical device may be used, for example, as an occlusion device or for the treatment of an aneurysm.

In some embodiments, an implantable medical device comprises a lattice framework having a metal core and a plurality of interconnected polymeric electrospun fiber members deposited on the metal core; and a polymeric electrospun mesh contacting at least two of the plurality of interconnected polymeric electrospun fiber members and having a pore size, wherein the medical device has an expanded configuration comprising an expanded diameter and a length, and a collapsed configuration comprising a collapsed diameter and a length, and wherein the expanded diameter is greater than the collapsed diameter.

In some embodiments, the length in the expanded configuration is less than or equal to the length in the collapsed configuration. In some embodiments, the polymeric electrospun mesh contacts at least two adjacent interconnected polymeric electrospun fiber members. In some embodiments, the metal core comprises a drawn filled tubing wire. In some embodiments, the polymeric electrospun mesh comprises a blend of at least two polymers. In some embodiments, the polymeric electrospun fiber members comprise a blend of at least two polymers. In some embodiments, the polymeric electrospun mesh covers at least one end of the medical device and is configured to occlude a blood vessel. In some embodiments, the polymeric electrospun mesh extends across the lattice framework covering openings between the plurality of polymeric electrospun fiber members. In some embodiments, the polymeric electrospun mesh covers a second end of the medical device. In some embodiments, the lattice framework comprises a repeating pattern in a shape of: bricks, hexagons, fish scales, vertical circles, horizontal circles, vertical diamonds, horizontal diamonds, vertical zig-zags, horizontal zig-zags, vertical sinusoids, or horizontal sinusoids. In some embodiments, at least one of the polymeric electrospun fiber members, the polymeric electrospun mesh, or the metal core comprises a contrast agent. In some embodiments, the lattice framework comprises a substantially tubular shaped, a substantially cone shaped, or a substantially bow-tie shaped configuration and is configured to occlude a blood vessel. In some embodiments, the pore size of the polymeric electrospun mesh is from about 5 μm to about 500 μm. In some embodiments, the pore size of the polymeric electrospun mesh is configured to remain constant as the implantable medical device changes between the expanded diameter and the collapsed diameter. In some embodiments, a density (e.g., porosity or space between fibers) of the polymeric electrospun mesh is configured to remain constant as the implantable medical device changes between the expanded diameter and the collapsed diameter. In some embodiments, a polymer solution is added to the metal core prior to depositing the polymeric electrospun fibers onto the metal core. In some embodiments, the plurality of interconnected polymeric electrospun fiber members comprise a porous architecture mimicking an extracellular matrix of tissue surrounding an implant site. In some embodiments, the lattice framework is configured to divert fluid flow through a blood vessel away from a patient aneurysm. In some embodiments, a pore size of the mesh is configured to prevent fluid flow therethrough.

In another embodiment, an embolization device configured to be inserted into and conform to a shape of an aneurysm of a patient, the embolization device comprises a coil having a substantially tubular metal core and a plurality of interconnected polymeric electrospun fiber members deposited on the substantially tubular metal core, the metal core comprising a drawn filled tubing wire, and a hydrophilic component. Such hydrophilic components and associated linkers are described in, for example, U.S. Pat. Pub. No. 2017/0071607, which is incorporated herein by reference in its entirety.

In some embodiments, the embolization device has an expanded configuration comprising an expanded diameter and an expanded length, and a collapsed configuration comprising a collapsed diameter and a collapsed length, and wherein the expanded diameter is greater than the collapsed diameter. In some embodiments, the embolization device further comprises a polymeric electrospun mesh contacting at least two of the plurality of interconnected polymeric electrospun fiber members and having a pore size. In some embodiments, at least one of the polymeric electrospun fiber members or the metal core comprises a contrast agent. In some embodiments, a polymer solution is added to the metal core prior to depositing the polymeric electrospun fibers onto the metal core. In some embodiments, the plurality of interconnected polymeric electrospun fiber members comprise a porous architecture mimicking an extracellular matrix of tissue surrounding an implant site.

The instant disclosure further relates to methods of manufacturing a medical device. The method includes providing a mandrel and a polymer injection system at a distance from the mandrel, and applying a charge to one or more of the mandrel and the polymer injection system. The polymer injection system is loaded with a polymer solution. The mandrel is spun at a rotation speed while the polymer solution is ejected from the polymer injection system at a flow rate to form a tubular section on the mandrel. After it is formed, the tubular section is removed from the mandrel and processed. In one embodiment, the method is used to form a stent device. In another embodiment, the method is used to form a flow diverter. In some embodiments, a metal lattice framework is positioned on the mandrel prior to ejecting the polymer solution. In another embodiment, a first portion of the polymer solution is applied to the metal lattice framework before the metal lattice framework is placed on the mandrel.

Some embodiments are directed to a method of manufacturing an implantable medical device comprising applying a charge to at least one of a mandrel or a polymer injection system, the polymer injection system spaced apart from the mandrel at a distance; loading the polymer injection system with a polymer solution; providing a metal core material on the mandrel; spinning the mandrel at a rotation speed; ejecting the polymer solution at a flow rate to deposit polymeric electrospun fiber members onto the metal core material on the mandrel; removing the metal core material with the deposited polymeric electrospun fiber members from the mandrel; and processing the removed metal core material with the deposited polymeric electrospun fiber members.

In some embodiments, the metal core material comprises a lattice framework on the mandrel. In some embodiments, the processing comprises laser cutting the removed metal core material with the deposited polymeric electrospun fiber members to form a lattice framework having a plurality of interconnected polymeric electrospun fiber members. In some embodiments, the method further comprises contacting a polymeric electrospun mesh to at least two adjacent polymeric electrospun fiber members to substantially cover openings between the adjacent polymeric electrospun fibers, the polymeric electrospun mesh having a pore size. In some embodiments, the metal material comprises a drawn filled tubing wiring. In some embodiments, a portion of the polymer solution is applied to the metal material prior to ejecting the polymer solution at a flow rate to deposit the polymeric electrospun fiber members onto the metal material. In some embodiments, the processing comprises at least one of: a dip-coating treatment, a heat treatment, or a solvent treatment. In some embodiments, the medical device is one of: a flow diverter, a vascular plug, or an embolization coil.

Further embodiments of the instant disclosure are described herein.

### BRIEF DESCRIPTION OF THE DRAWINGS

FIG. 1A is a scanning electron microscope (SEM) image of a tubular electrospun fiber section without a post-manufacturing treatment, in accordance with the instant disclosure.

FIG. 1B is an SEM image of the tubular electrospun fiber section of FIG. 1A after undergoing a post-manufacturing treatment in isopropanol (IPA) in accordance with the instant disclosure.

FIG. 2 is plot of plate compression data for a tubular electrospun fiber section made from PGLA 82:18 in accordance with the instant disclosure.

FIG. 3A is an SEM image of a stent design laser cut from a tubular electrospun fiber section in accordance with the instant disclosure.

FIG. 3B is an SEM image of a stent design laser cut from a tubular electrospun fiber section in accordance with the instant disclosure.

FIG. 4A illustrates a brick lattice framework pattern in accordance with the instant disclosure.

FIG. 4B illustrates a hexagon lattice framework pattern in accordance with the instant disclosure.

FIG. 4C illustrates a fish scale lattice framework pattern in accordance with the instant disclosure.

FIG. 4D illustrates a star lattice framework pattern in accordance with the instant disclosure.

FIG. 5A illustrates a vertical circle lattice framework pattern in accordance with the instant disclosure.

FIG. 5B illustrates a horizontal circle lattice framework pattern in accordance with the instant disclosure.

FIG. 5C illustrates a vertical diamond lattice framework pattern in accordance with the instant disclosure.

FIG. 5D illustrates a horizontal diamond lattice framework pattern in accordance with the instant disclosure.

FIG. 6A illustrates a vertical zig-zag lattice framework pattern in accordance with the instant disclosure.

FIG. 6B illustrates a horizontal zig-zag lattice framework pattern in accordance with the instant disclosure.

FIG. 6C illustrates a vertical sinusoid lattice framework pattern in accordance with the instant disclosure.

FIG. 6D illustrates a horizontal sinusoid lattice framework design in accordance with the instant disclosure.

FIG. 7A illustrates a flow diverter in accordance with the instant disclosure.

FIG. 7B illustrates a flow diverter in a compressed or delivery configuration in accordance with the instant disclosure.

FIG. 8 illustrates a substantially tubular lattice framework comprising about 16 wire strands formed into a plurality of interconnected members, in accordance with the instant disclosure, and shows an embodiment of the framework being bent and manipulated while maintaining flexibility and kink resistance.

FIG. 9A illustrates a flow diverter having a substantially tubular lattice framework comprising about 16 wire strands formed into a plurality of interconnected members, in accordance with the instant disclosure.

FIG. 9B illustrates the flow diverter of FIG. 9A formed into a curve, in accordance with the instant disclosure.

FIG. 9C illustrates an alternative view of the flow diverter of FIG. 9A formed into a curve, in accordance with the instant disclosure.

FIG. 10A illustrates a flow diverter comprising about 32 wire strands formed into a plurality of interconnected members, in accordance with the instant disclosure.

FIG. 10B illustrates the flow diverter of FIG. 10A formed into a curve, in accordance with the instant disclosure.

FIG. 10C illustrates an alternative view of the flow diverter of FIG. 10A formed into a curve, in accordance with the instant disclosure.

FIG. 11A illustrates a flow diverter comprising about 48 wire strands formed into a plurality of interconnected members, in accordance with the instant disclosure.

FIG. 11B illustrates the flow diverter of FIG. 11A formed into a curve, in accordance with the instant disclosure.

FIG. 11C illustrates an alternative view of the flow diverter of FIG. 11A formed into a curve, in accordance with the instant disclosure.

FIG. 12A illustrates a schematic longitudinal cross section of a medical device comprising a lattice framework and a polymeric electrospun mesh covering at least one end, in accordance with the present disclosure.

FIG. 12B illustrates a schematic side view of a medical device comprising a lattice framework and a polymeric electrospun mesh covering at least one end, in accordance with the present disclosure.

FIG. 12C illustrates a schematic side view of a medical device comprising a lattice framework and a polymeric electrospun mesh covering at least one end, in accordance with the present disclosure.

FIG. 12D illustrates a schematic side view of a medical device comprising a lattice framework and a polymeric electrospun mesh covering at least one end, the medical device having a bowtie shape and including a marker, in accordance with the present disclosure.

FIG. 12E illustrates a schematic side view of a medical device comprising a lattice framework and a polymeric electrospun mesh covering at least one end, the medical device having a cone shape and including a marker, in accordance with the present disclosure.

FIG. 13A illustrates schematic side view of a intrasaccular device in an expanded configuration positioned within an aneurysm such that it conforms to a body and/or neck portion of the aneurysm to substantially occlude blood or other fluid flow into the aneurysm.

FIG. 13B illustrates schematic side view of the intrasaccular device in a compressed configuration.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

The instant disclosure relates to medical devices. In some embodiments, the medical device is a stent device. In other embodiments, the medical device is a flow diverter. In still other embodiments, the medical device may be used in the sac of an aneurysm (i.e. “intrasaccular”), while in certain embodiments, the medical device may be used to occlude a vessel or aneurysm. The medical devices disclosed herein may be formed with or include polymeric electrospun fibers. In some embodiments, the polymeric electrospun fibers are electrospun into substantially tubular structures, which are further processed to produce a plurality of interconnected polymeric electrospun fiber members. In other embodiments, the medical device may take on a shape that is not substantially tubular, but is instead a bowtie or cone shape, as described herein.

### Electrospinning Fibers

Electrospinning is a method which may be used to process a polymer solution into a fiber. In embodiments wherein the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold 101 may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate and the polymer may stretch, leaving a long, thin fiber that is deposited onto the mandrel. The polymer solution may form a Taylor cone as it is ejected from the polymer injection system and exposed to a charge.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components, with each syringe containing the same polymer solution. In some embodiments, a multi-syringe polymer injection system may include multiple syringes and multiple needles or needle-like components, with each syringe containing a different polymer solution. In some embodiments, a charge may be applied to the polymer injection system, or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.5 μm, about 1 μm, about 2 μm, about 5 μm, about 10 μm, about 20 μm, or ranges between any two of these values, including endpoints.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

The non-resorbable polymers may include, in some non-limiting examples, polyethylene, polyethylene oxide, polyethylene terephthalate, polyester, polymethylmethacrylate, polyacrylonitrile, silicone, polyurethane, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, copolymers thereof, or combinations thereof.

The resorbable polymers may include, in some non-limiting examples, polycaprolactone, poly(lactide-co-caprolactone), poly(lactide-co-glycolide), polyglycolide, polylactic acid, including derivatives thereof such as, without limitation, poly(L-lactic acid), and poly(D, L-lactic acid), polyglycolic acid, polydioxanone, poly(-hydroxybutyrate-co-3-hydroxyvalerate), trimethylene carbonate, polydiols, polyesters, polyethylene terephthalate, polyurethane, polyethylene, polyethylene oxide, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polyglycolic acid, polydioxanone, collagen, gelatin, fibrin, fibronectin, albumin, hyaluronic acid, elastin, chitosan, alginate, or combinations thereof. In one embodiment, the resorbable polymers are selected from poly(lactide-co-glycolide), polyglycolide, poly(L-lactic acid), copolymers thereof, and combinations thereof. In one embodiment, the resorbable polymer comprises poly(lactide-co-glycolide). In another embodiment, the resorbable polymer comprises poly(L-lactic acid).

The natural polymers may include, in some non-limiting examples, collagen, gelatin, fibrin, fibronectin, albumin, hyaluronic acid, elastin, chitosan, alginate, silk, copolymers thereof, or combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed or more than one type of polymer, and that each polymer therein may have a specific characteristic, such as bio-stability or biodegradability.

### Applying Charges to Electrospinning Components

In an electrospinning system, one or more charges may be applied to one or more components, or portions of components, such as, for example, a mandrel or a polymer injection system, or portions thereof. In some embodiments, a positive charge may be applied to the polymer injection system, or portions thereof. In some embodiments, a negative charge may be applied to the polymer injection system, or portions thereof. In some embodiments, the polymer injection system, or portions thereof, may be grounded. In some embodiments, a positive charge may be applied to mandrel, or portions thereof. In some embodiments, a negative charge may be applied to the mandrel, or portions thereof. In some embodiments, the mandrel, or portions thereof, may be grounded. In some embodiments, one or more components or portions thereof may receive the same charge. In some embodiments, one or more components, or portions thereof, may receive one or more different charges.

The charge applied to any component of the electrospinning system, or portions thereof, may be from about −15 kV to about 30 kV, including endpoints. In some non-limiting examples, the charge applied to any component of the electrospinning system, or portions thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or portions thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Stent Device

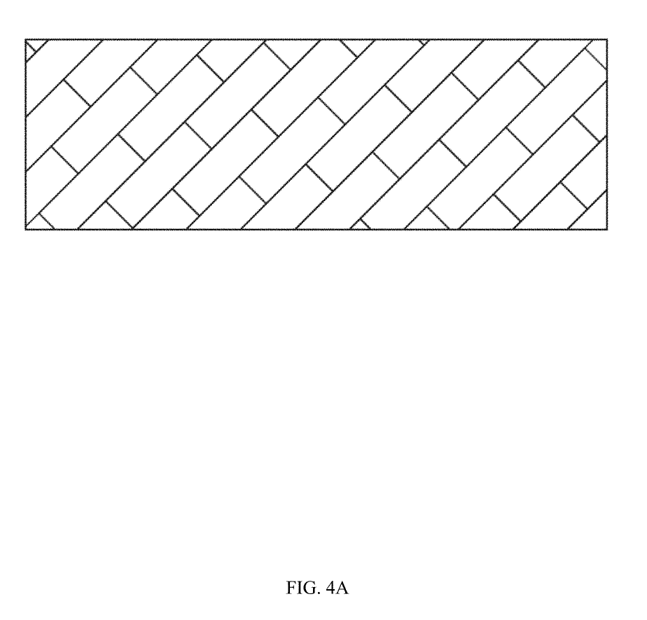

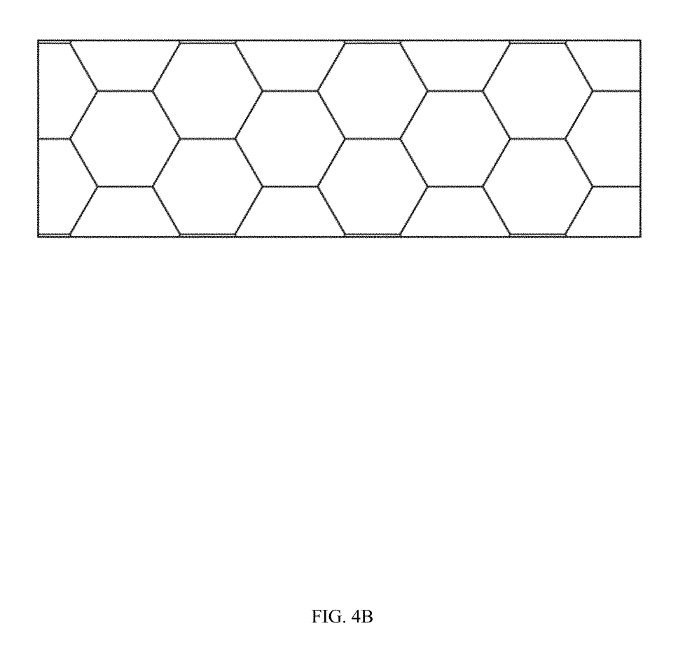

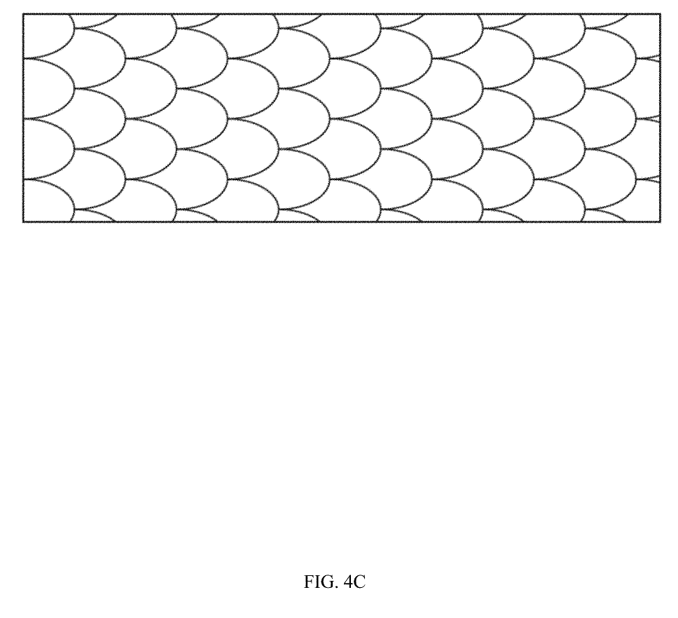

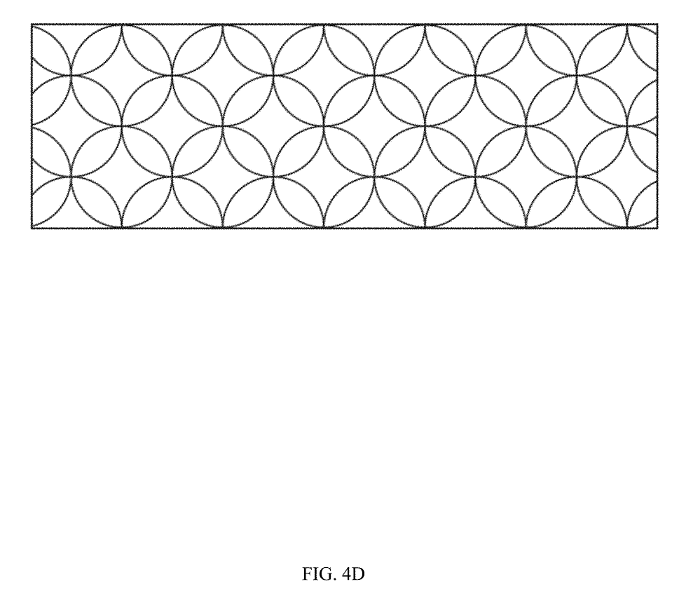

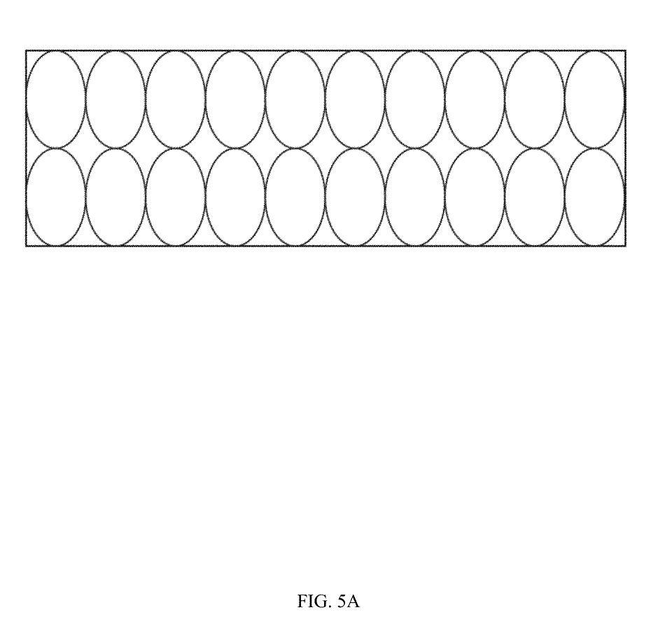

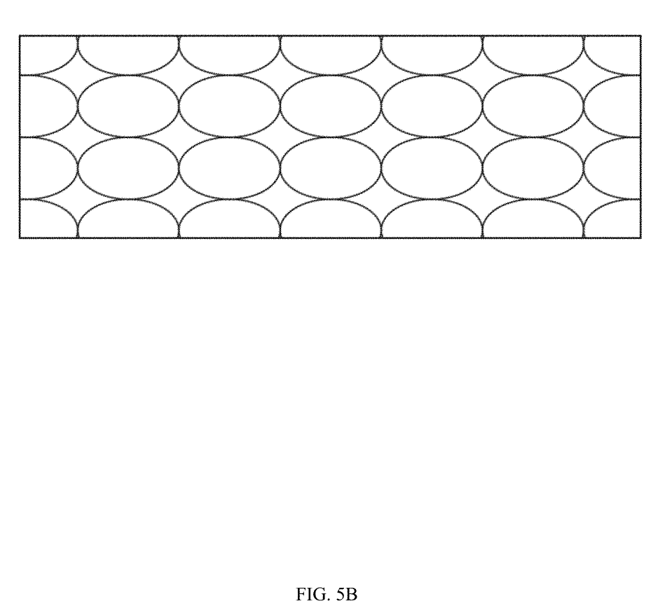

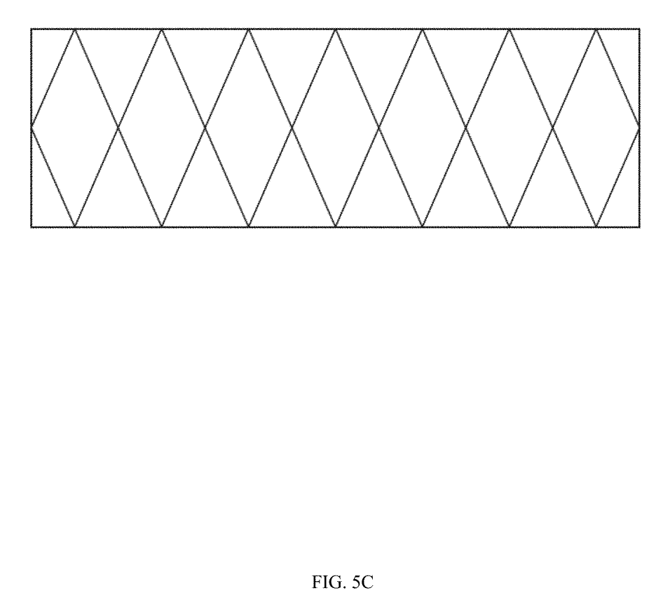

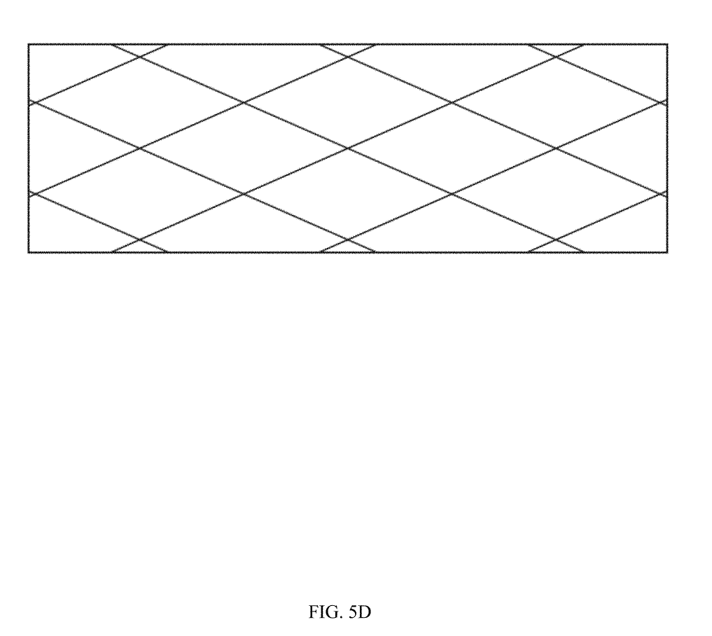

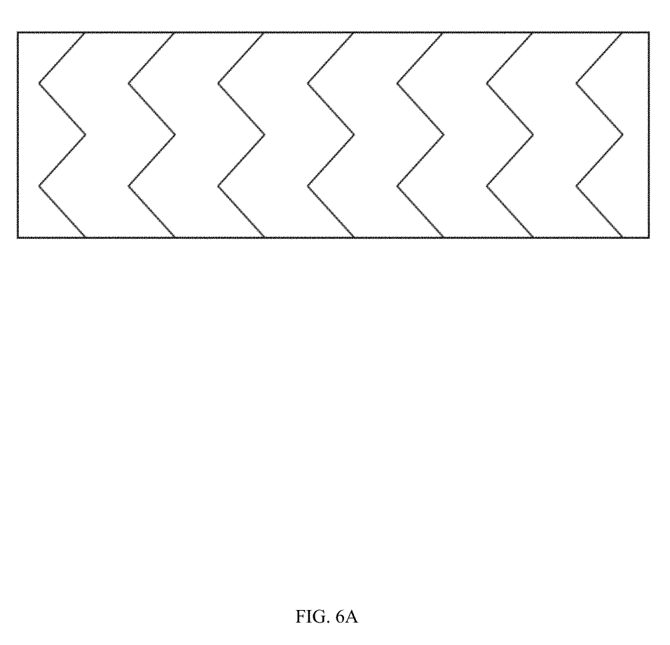

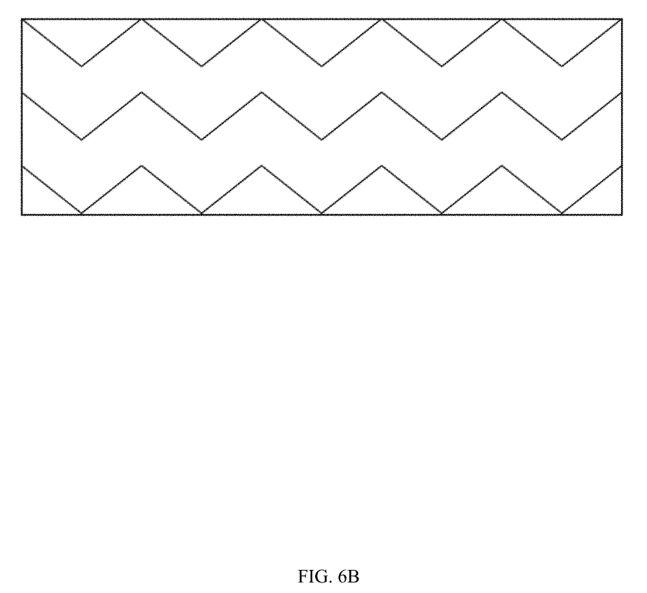

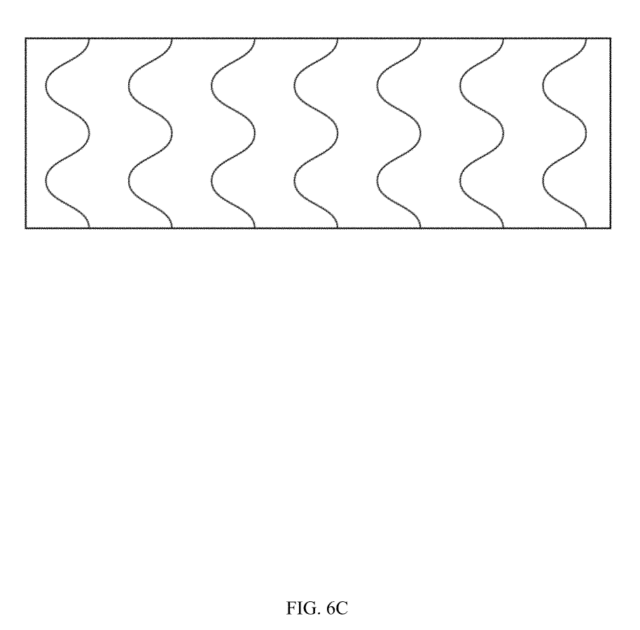

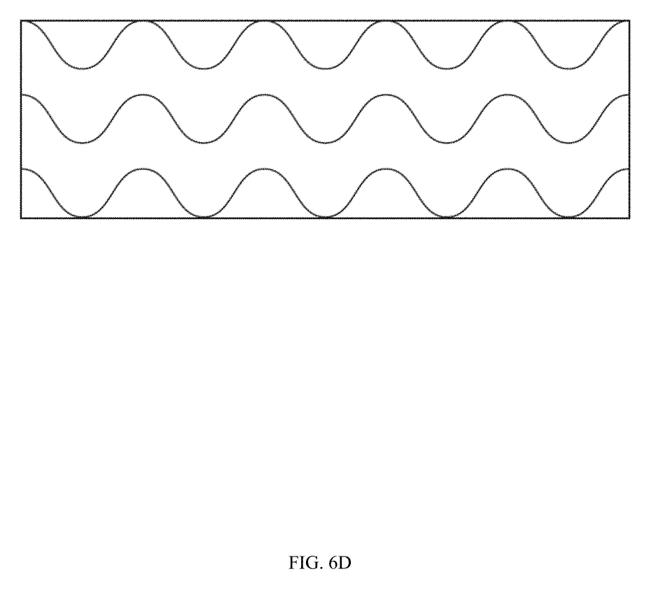

In one embodiment, the polymeric electrospun fibers may be used to form a stent device. In an embodiment, a stent device may include a substantially tubular lattice framework 101 having a plurality of interconnected polymeric electrospun fiber members. In another embodiment, a stent device may include a substantially tubular lattice framework 101 having at least one wire strand formed into a plurality of interconnected members. The lattice framework 101 may include any random or repeating pattern as would be apparent to those of skill in the art in view of this disclosure. Suitable patterns include, without limitation, bricks, hexagons, fish scales, stars, vertical circles, horizontal circles, vertical diamonds, horizontal diamonds, vertical zig-zags, horizontal zig-zags, vertical sinusoids, horizontal sinusoids, and the like. Examples of bricks, hexagons, fish scales, stars, vertical circles, horizontal circles, vertical diamonds, horizontal diamonds, vertical zig-zags, horizontal zig-zags, vertical sinusoids, and horizontal sinusoids for the lattice framework 101 patterns are illustrated in FIG. 4A, FIG. 4B, FIG. 4C, FIG. 4D, FIG. 5A, FIG. 5B, FIG. 5C, FIG. 5D, FIG. 6A, FIG. 6B, FIG. 6C, and FIG. 6D, respectively.

In some embodiments, the substantially tubular lattice framework 101 of a stent device may comprise at least one wire strand formed into a plurality of interconnected members. In some embodiments, the at least one wire strand may be braided to form a plurality of interconnected members. In other embodiments, the at least one wire strand may be folded or woven to form a plurality of interconnected members. In certain embodiments, the substantially tubular lattice framework 101 may comprise from about 1 wire strand to about 64 wire strands. In some embodiments, the substantially tubular lattice framework 101 may comprise, for example, about 1 wire strand, about 2 wire strands, about 4 wire strands, about 6 wire strands, about 8 wire strands, about 10 wire strands, about 12 wire strands, about 14 wire strands, about 16 wire strands, about 18 wire strands, about 20 wire strands, about 22 wire strands, about 24 wire strands, about 26 wire strands, about 28 wire strands, about 30 wire strands, about 32 wire strands, about 34 wire strands, about 36 wire strands, about 38 wire strands, about 40 wire strands, about 42 wire strands, about 44 wire strands, about 46 wire strands, about 48 wire strands, about 50 wire strands, about 52 wire strands, about 54 wire strands, about 56 wire strands, about 58 wire strands, about 60 wire strands, about 62 wire strands, about 64 wire strands, or any range between any two of these values, including endpoints. In some embodiments, the at least one wire of a substantially tubular lattice framework 101 may comprise a metal such as, for example, stainless steel, gold, titanium, cobalt, chromium, tantalum, nickel, titanium, magnesium, iron, alloys thereof, and combinations thereof. In some embodiments, the at least one wire of a substantially tubular lattice framework 101 may comprise a drawn-filled tubing (DFT) wire. DFT wire, as described herein, may include outer layers or materials that are substantially corrosion resistant. For example, such outer layers or materials may include nickel titanium, platinum iridium, or gold. Further, in accordance with certain embodiments, the DFT wire as described herein may range in diameter from about 0.0005 inches to about 0.005 inches.

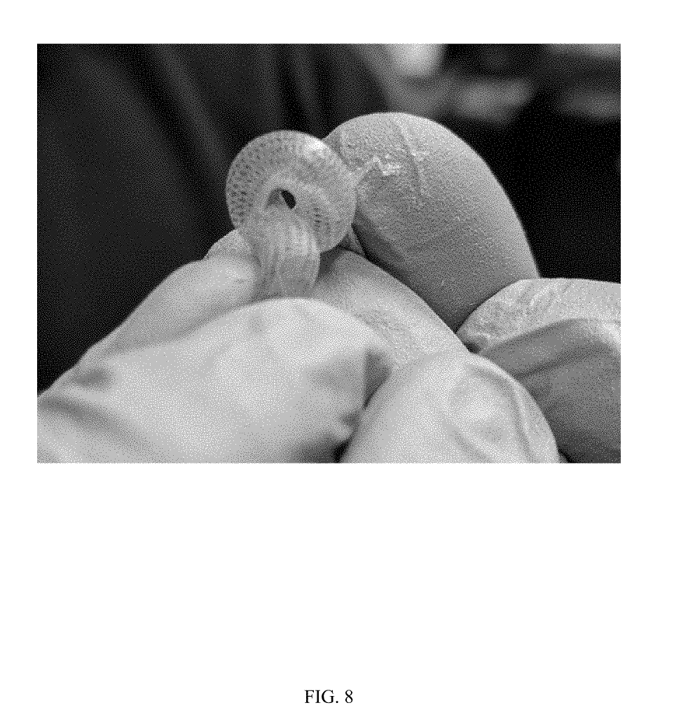

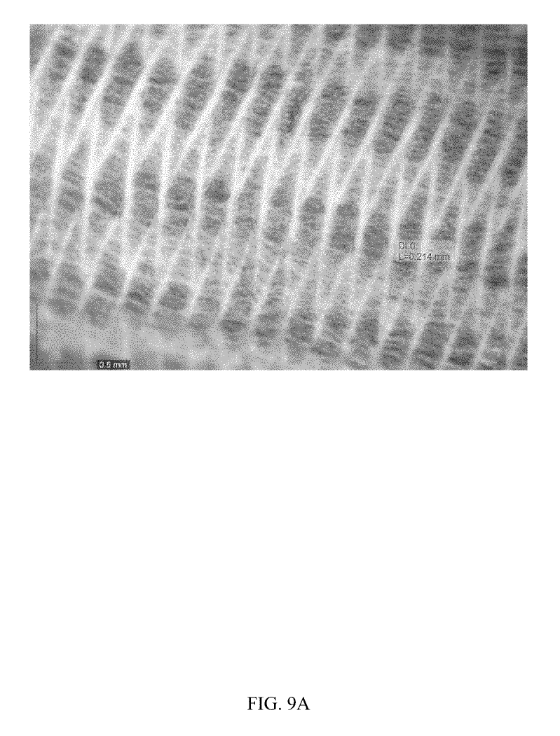

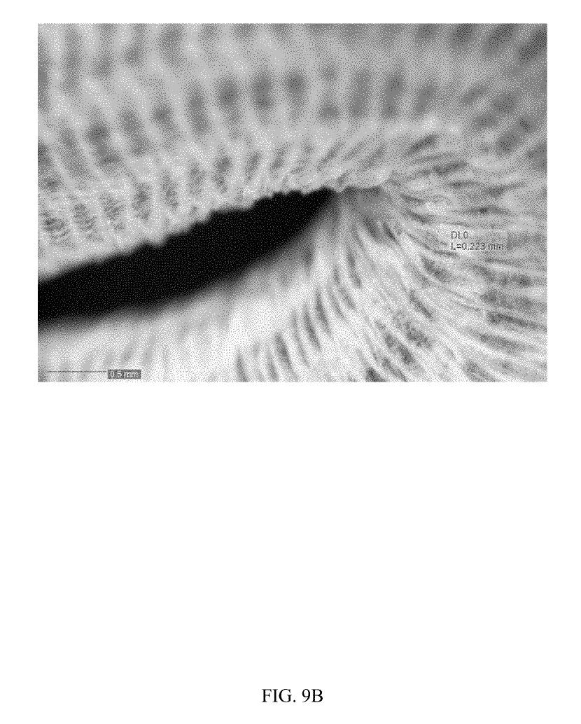

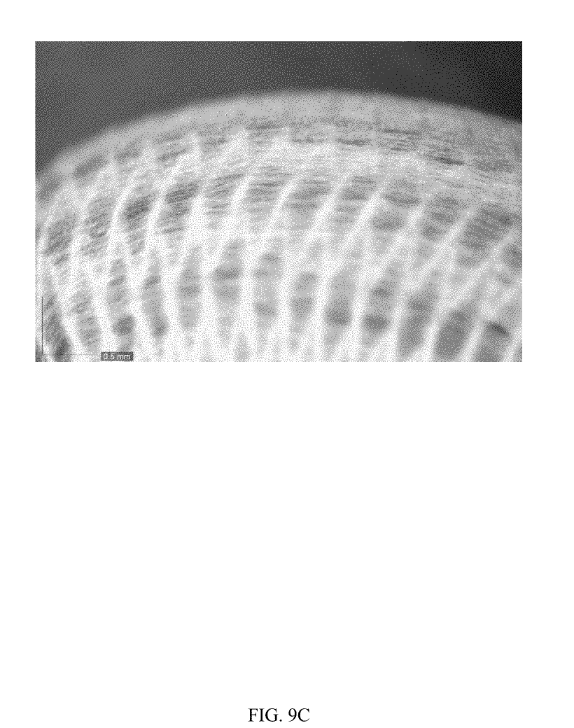

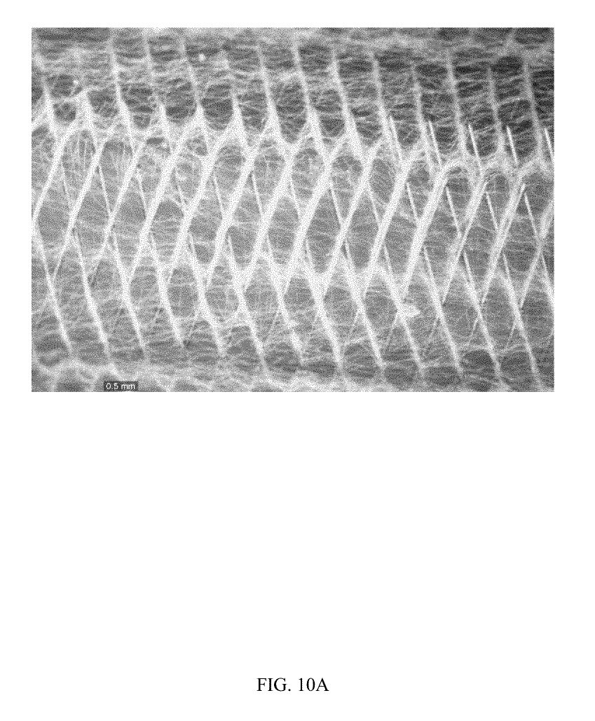

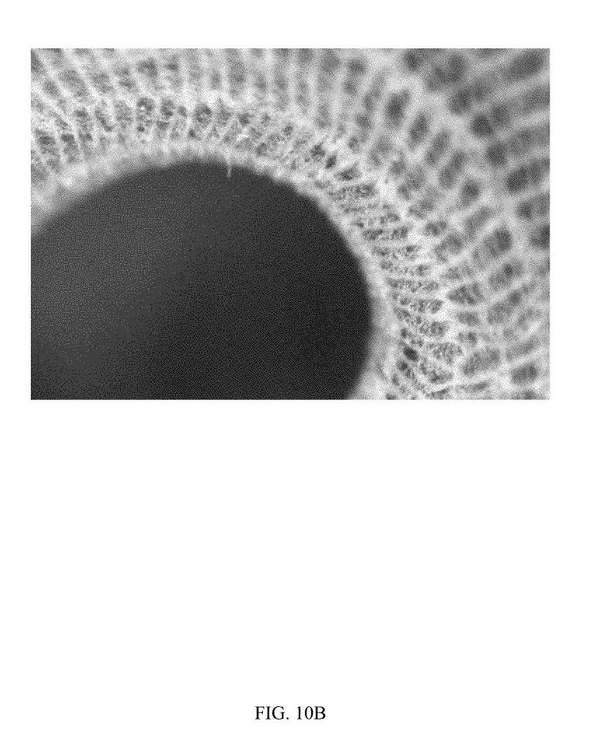

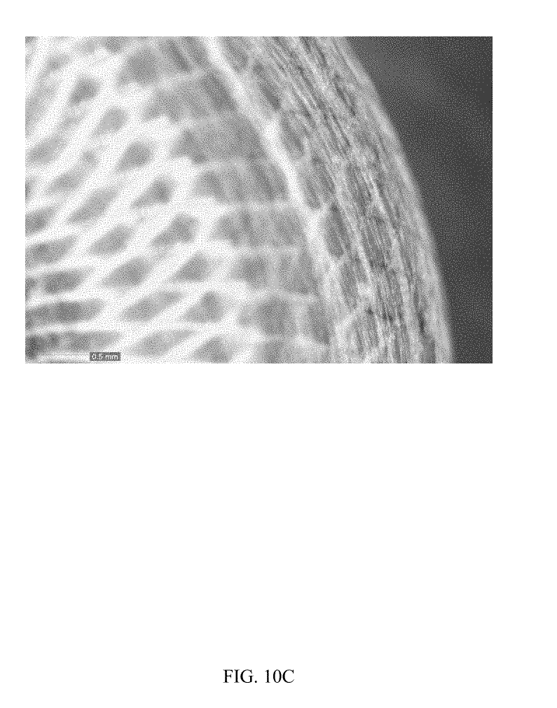

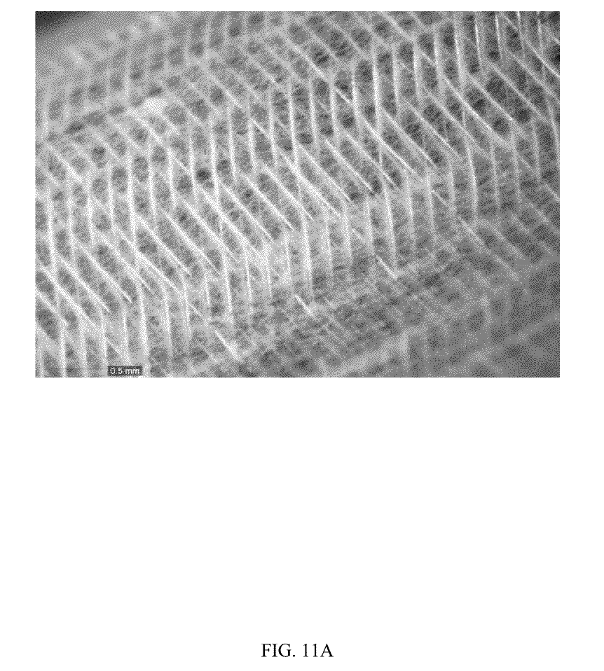

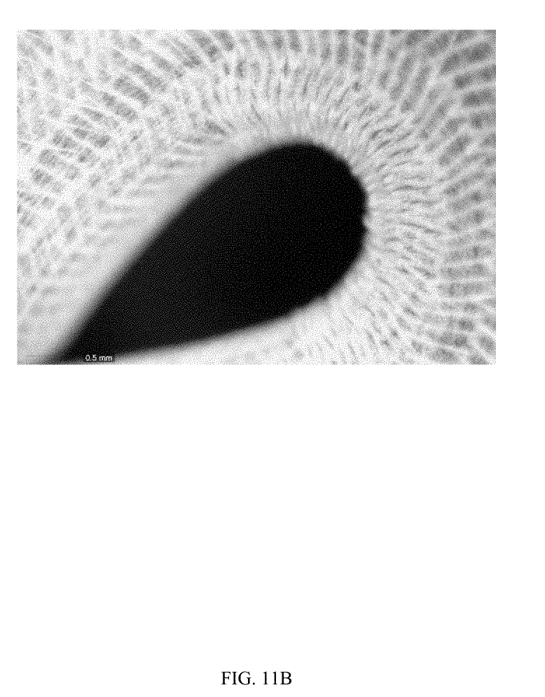

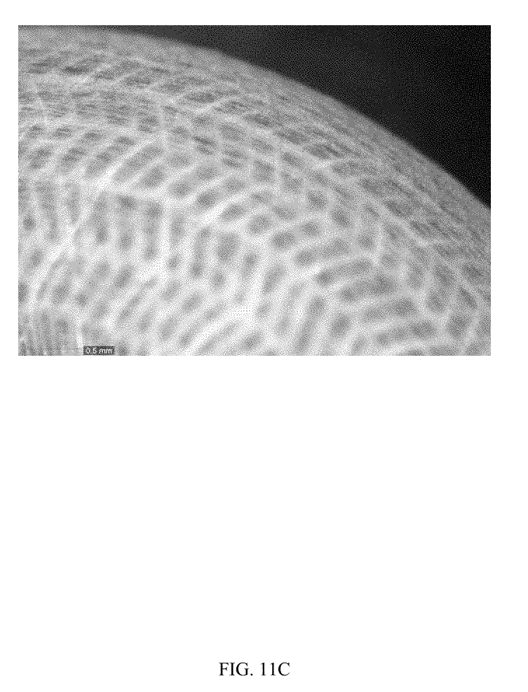

In certain embodiments, a substantially tubular lattice framework 101 having at least one wire strand formed into a plurality of interconnected members may serve to maintain the flexibility of a stent, flow diverter, or other medical device by allowing the device to be bent and curved without kinking, as illustrated in FIG. 8, FIG. 9A, FIG. 9B, FIG. 9C, FIG. 10A, FIG. 10B, FIG. 10C, FIG. 11A, FIG. 11B, and FIG. 11C. In particular, FIG. 9A, FIG. 9B, and FIG. 9C illustrate the flexibility of a substantially tubular lattice framework 101 comprising 16 wire strands formed into a plurality of interconnected members. Similarly, FIG. 10A, FIG. 10B, and FIG. 10C illustrate the flexibility of a substantially tubular lattice framework 101 comprising 32 wire strands formed into a plurality of interconnected members. Moreover, FIG. 11A, FIG. 11B, and FIG. 11C illustrate the flexibility of a substantially tubular lattice framework 101 comprising 48 wire strands formed into a plurality of interconnected members.

The stent device may further include an operational configuration having an operational diameter and operational length, and a delivery configuration having a delivery diameter and delivery length. As used herein, operational configuration refers to an operational diameter and operational length of the stent device after it has been inserted into a patient. Further, as used herein delivery configuration refers to a delivery diameter and delivery length of the stent device during the delivery or placement of the stent. For example, the stent device may be delivered into a patient using a balloon catheter where the stent device surrounds a deflated balloon, and is in a compressed or delivery configuration. Once the stent device is positioned in its desired location, such as, for example, a coronary artery, the balloon may be inflated to expand the stent device into an operational configuration. Alternatively, a stent device may be stretched into its delivery configuration, and delivered into a patient using a microcatheter; once the stretched stent device reaches the correct position within the patient, it may be released from the microcatheter and self-expand into its operational configuration. In some embodiments, the medical device disclosed herein may be easily repositioned even after it has self-expanded into its operational configuration. In an embodiment, such repositioning may be accomplished, for example, by retracting the device back into the microcatheter (“resheathing” the device), repositioning the device, and then allowing the device to expand to its operational configuration again. In one embodiment, the operational diameter is greater than the delivery diameter. In another embodiment, the operational length is less than or equal to the delivery length. In one embodiment, the operational length is greater than the delivery length.

In some embodiments, the ratio of the delivery length to the operational length is from about 1:1 to about 2:1. The ratio of the delivery length may be, for example, about 1:1, about 1.1:1, about 1.2:1, about 1.3:1, about 1.4:1, about 1.5:1, about 1.6:1, about 1.7:1, about 1.8:1, about 1.9:1, about 2:1, or any range between any two of these ratios, including endpoints. In one embodiment, the ratio of the delivery length to the operational length is about 1:1. Without wishing to be bound by theory, a ratio of the delivery length to the operational length from about 1:1 to about 1.7:1 may be accomplished by the interconnected electrospun polymer fiber members collapsing upon themselves, and later expanding, without a significant change in length or diameter between the collapsed (“delivery”) and expanded (“operational”) configurations.

In some embodiments, the operational length may be from about 1 cm to about 15 cm. The operational length may be, for example, about 1 cm, about 1.5 cm, about 2 cm, about 2.5 cm, about 3 cm, about 3.5 cm, about 4 cm, about 4.5 cm, about 5 cm, about 5.5 cm, about 6 cm, about 6.5 cm, about 7 cm, about 7.5 cm, about 8 cm, about 8.5 cm, about 9 cm, about 9.5 cm, about 10 cm, about 10.5 cm, about 11 cm, about 11.5 cm, about 12 cm, about 12.5 cm, about 13 cm, about 13.5 cm, about 14 cm, about 14.5 cm, about 15 cm, or any range between any two of these values, including endpoints.

In some embodiments, the delivery length may be from about from about 1 cm to about 30 cm. The delivery length may be, for example, about 1 cm, about 1.5 cm, about 2 cm, about 2.5 cm, about 3 cm, about 3.5 cm, about 4 cm, about 4.5 cm, about 5 cm, about 5.5 cm, about 6 cm, about 6.5 cm, about 7 cm, about 7.5 cm, about 8 cm, about 8.5 cm, about 9 cm, about 9.5 cm, about 10 cm, about 10.5 cm, about 11 cm, about 11.5 cm, about 12 cm, about 12.5 cm, about 13 cm, about 13.5 cm, about 14 cm, about 14.5 cm, about 15 cm, about 15.5 cm, about 16 cm, about 16.5 cm, about 17 cm, about 17.5 cm, about 18 cm, about 18.5 cm, about 19 cm, about 19.5 cm, about 20 cm, about 20.5 cm, about 21 cm, about 21.5 cm, about 22 cm, about 22.5 cm, about 23 cm, about 23.5 cm, about 24 cm, about 24.5 cm, about 25 cm, about 25.5 cm, about 26 cm, about 26.5 cm, about 27 cm, about 27.5 cm, about 28 cm, about 28.5 cm, about 29 cm, about 29.5 cm, about 30 cm, or any range between any two of these values, including endpoints.

In some embodiments, the delivery diameter may be from about 0.011 inches to about 0.026 inches. The delivery diameter may be, for example, about 0.011 inches, about 0.012 inches, about 0.013 inches, about 0.014 inches, about 0.015 inches, about 0.016 inches, about 0.017 inches, about 0.018 inches, about 0.019 inches, about 0.02 inches, about 0.021 inches, about 0.022 inches, about 0.023 inches, about 0.024 inches, about 0.025 inches, about 0.026 inches, or any range between any two of these values, including endpoints. In one embodiment, the delivery diameter is about 0.02 inches.

In some embodiments, the operational diameter may be from about 2 mm to about 9 mm. The operational diameter may be, for example, about 2 mm, about 3 mm, about 4 mm, about 5 mm, about 6 mm, about 7 mm, about 8 mm, about 9 mm, or any range between any two of these values, including endpoints. In one embodiment, the operational diameter is about 4 mm.

In some embodiments, a contrast agent may be included in the polymeric electrospun fiber members. Inclusion of contrast agents allows the stent device to be viewed and/or monitored with standard radiological imaging techniques, such as, for example, fluoroscopic imaging, during and following its insertion into a vessel. In some embodiments, the contrast agent may be, for example, bismuth, bismuth (III) oxide, barium, tungsten, iodine, gadolinium, gold, platinum, tantalum, gadolinium, diatrizoate, metrizoate, ioxaglate, iopamidol, iohexol, ioxilan, iopromide, iodixanol, or any combination thereof. In one embodiment, the polymeric solution comprises the contrast agent. In another embodiment, the contrast agent is added to the stent device post-fabrication.

In some embodiments, the stent device may include a hydrophilic component which may allow the device to be more easily deployed by allowing it to pass easily though a catheter or other delivery vehicle. In some embodiments, the hydrophilic component may be, for example, a polymer.

In some embodiments, the interconnected polymeric electrospun fiber members which form the lattice framework 101 may have a porous architecture. Such an architecture may mimic the extracellular matrix (ECM) of the tissue surrounding the placement location of the stent device. The mimicking of the ECM is believed to permit cells to grow on the ECM and induce tissue regeneration leading to a rapid re-endothelialization at the interface between the stent device and the tissue surrounding the placement location. In another embodiment, the polymeric electrospun fiber members which form the lattice framework 101 may have a fibrous architecture. The polymeric electrospun fiber members may provide a regenerative advantage over solid polymer alternatives for resorbable stenting applications.

In one embodiment, the stent device is made from biodegradable or resorbable polymers that degrade after placement in a patient. In some embodiments, the stent device may substantially degrade over a time period ranging from about 3 months to about 24 months. In other embodiments, the stent device may degrade over a time period of about 5 months to about 20 months. In one embodiment, the stent device may degrade over a time period of about 6 to about 12 months. In some embodiments, the stent may degrade in any of 3 months, 4 months, 5 months, 6, months, 7 months, 8 months, 10 months, 12 months, 15 months, 20 months and 24 months. The amount of time in which the stent degrades in a patient may be dependent on the desired application, and such time periods would be apparent to one of skill in the art in view of this disclosure. The stent device can be configured to degrade over a desired time frame by adjusting the types of resorbable polymers used for the interconnected polymeric electrospun fiber members. For example, one resorbable polymer may be employed, providing a degrading time frame specific for the chosen polymer. In other embodiments, a mixture of resorbable polymers may be employed where the degradation time frame is staggered based on the degradation rates of the individual resorbable polymers.

In some embodiments, the stent device may further comprise a treatment. In one embodiment, the treatment may include one or more of a dip-coating treatment, a heat treatment, and a solvent treatment. In another embodiment, the solvent treatment comprises IPA. In some embodiments, the treatment may cause the stent to shrink from its originally fabricated size. Without limiting the scope of the instant disclosure, it is believed that the shrinkage caused by the treatment may cause a stiffening and/or strengthening of the lattice framework 101.

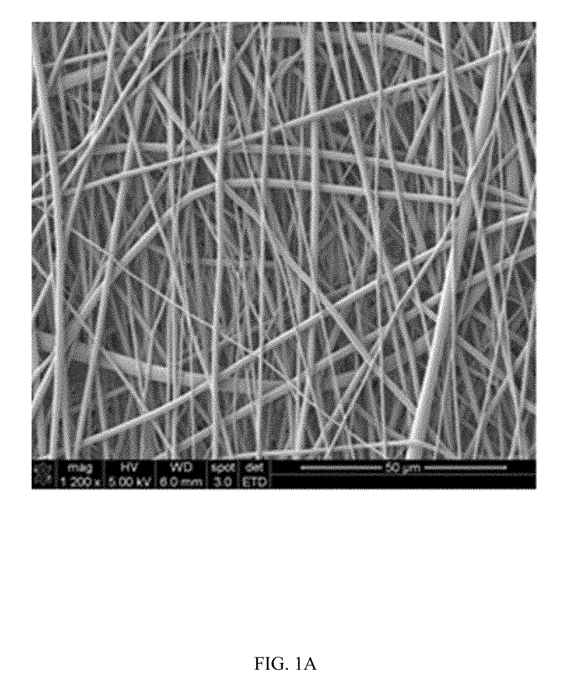

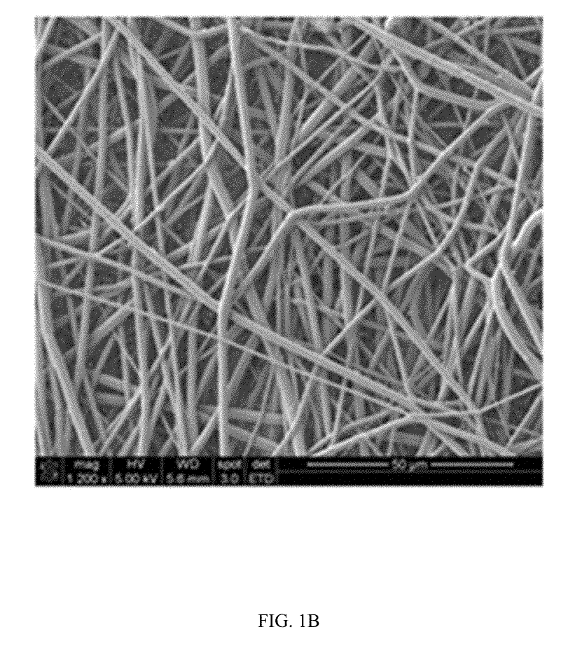

FIG. 1A is an SEM image of a section of lattice framework 101 of interconnected polymeric electrospun fiber members prior to an IPA treatment. FIG. 1B is an SEM image of the same section after an IPA treatment. The interconnected polymer electrospun fiber members of FIG. 1A were made from a poly(lactide-co-glycolide) having a weight ratio of D,L,-lactide to glycolide of about 82:18 (referred to herein as PGLA 82:18). The electrospinning parameters include a 15 kV (+11.1 kV/−4.0 kV), 5 ml/hr flow rate, 20 cm tip-to-collector distance, and a 6,500 rpm collector rotation speed. The electrospinning parameters resulted in bead-free fibers with few defects and increased stiffness of the polymeric electrospun fiber members upon treatment with IPA. The polymeric electrospun fibers in FIG. 1B have a “kinked” orientation that is likely an effect of the lattice framework 101 shrinking upon drying of the IPA. The fast rotation of the rod collector induced a substantially linear/parallel fiber arrangement around the circumference of the electrospun tube, which resulted in greater radial stiffness. The final electrospun tube can then be further processed to generate the lattice framework 101. In some embodiments, the lattice framework 101 may be laser-cut from the electrospun tube.

In some embodiments, the stent device may have a chronic outward force from about 0.1 N to about 10 N. The stent device may have a chronic outward force of, for example, about 0.1 N, about 0.5 N, about 1 N, about 2 N, about 3 N, about 4 N, about 5 N, about 6 N, about 7 N, about 8 N, about 9 N, about 10 N, or any range between any two of these values, including endpoints. In one embodiment, the stent device may have a chronic outward force of less than about 2.43 N.

In some embodiments, the stent device may have a radial resistive force from about 1 N to about 10 N. The stent device may have a radial resistive force of, for example, about 1 N, about 2 N, about 3 N, about 4 N, about 5 N, about 6 N, about 7 N, about 8 N, about 9 N, about 10 N, or any range between any two of these values, including endpoints. In one embodiment, the stent device may have a radial resistive force of less than about 20.3 N.

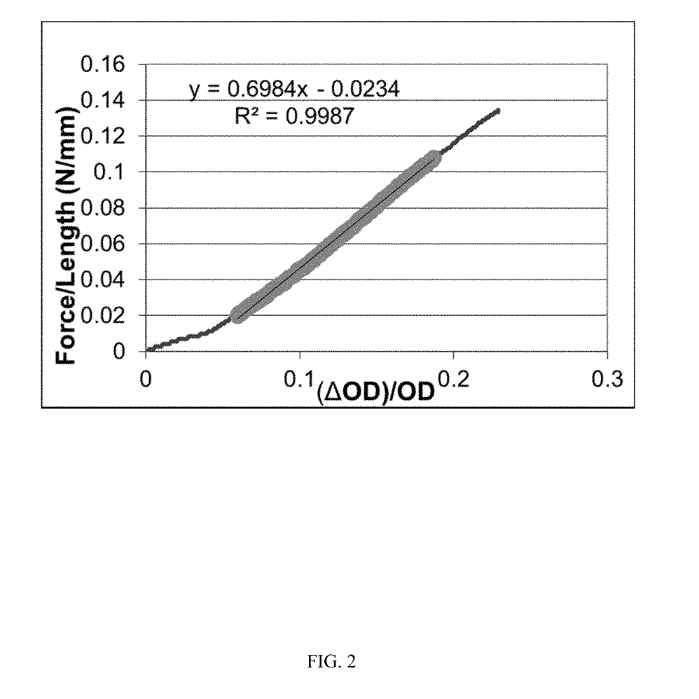

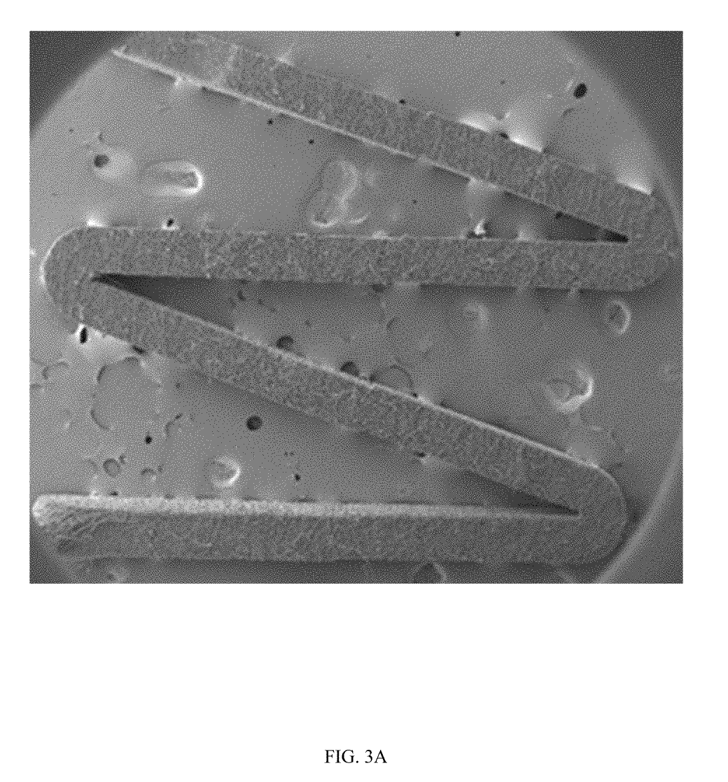

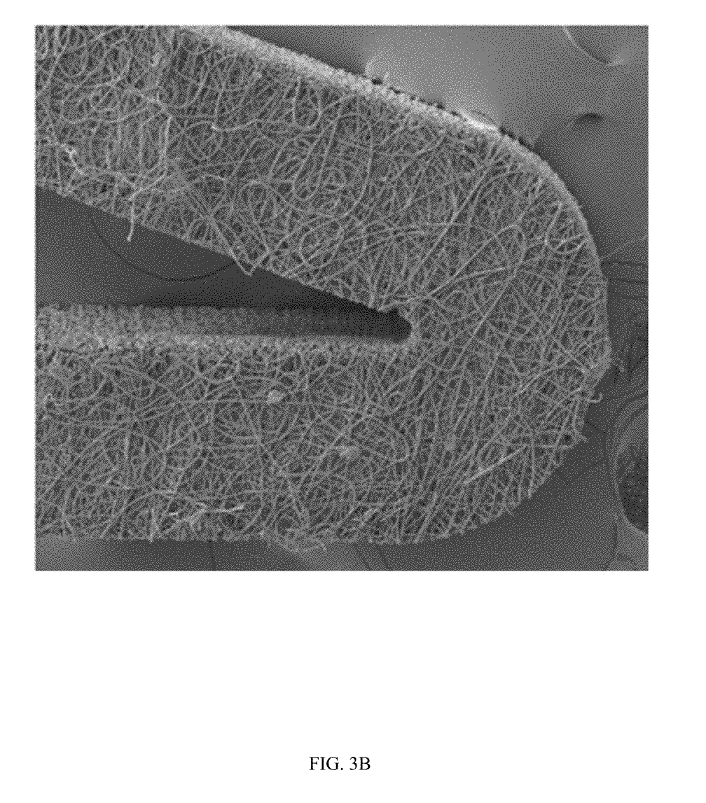

Radial stiffness of the electrospun tube may be measured by plate compression testing. The testing apparatus applies the pinching load to the electrospun tube by moving flat plates toward each other at a constant rate, while recording the force. FIG. 2 is a plot of the plate compression data for an electrospun tube made from PGLA 82:18. The plot in FIG. 2 charts force/length (N/mm) versus the change in the diameter of the electrospun tube under the pinching load divided by the original diameter (ΔOD/OD). The slope of the linear portion of the graph relates to the stiffness of the electrospun tube. Software was used to determine the pressures required to deform tested stents from a resting position to a crimped state. One suitable software program for such analysis includes Inventor® Finite Element Analysis software (Autodesk). Table 1 below describes five samples with varying designs for the lattice framework 101, or struts, of the stent device. The strut designs of samples 3 and 5 required the highest pressures to deform to 7F (2.33 mm). The strut designs of samples 3 and 5 were laser cut from a electrospun tube of PGLA 82:18, and examined under the SEM for any defects from the laser-cutting process. FIG. 3A and FIG. 3B show SEM images at varying magnifications of the resulting stent after the laser-cutting process. The SEM images indicate that the fibrous and porous architecture of the stents were maintained on the surfaces and edges, indicating that substantially no damage occurred during the laser-cutting process.

TABLE 1
Radial stiffness data

| | | | | Max Principle |
|---|---|---|---|---|
| Sample | w (mm) | θ (degrees) | Pressure at 2.33 mm | Stresses (MPa) |
| 1 | 0.2 | 10 | 0.0031 | 46.2 |
| 2 | 0.2 | 7 | 0.0009 | 19.9 |
| 3 | 0.2 | 20 | 0.0186 | 107.7 |
| 4 | 0.3 | 10 | 0.0004 | 53.93 |
| 5 | 0.1 | 10 | 0.0094 | 64.53 |

Radial stiffness of the stents of samples 3 and 5 were evaluated in accordance with ASTM F3067-14 for radial stiffness under a sling testing apparatus. The sling apparatus setup provides nearly uniform radial loading to measure chronic outward force (COF) and radial resistive force (RRF), both of which are standards for stiffness in self-expanding stents. The COF and RRF for samples 3 and 5 are shown in Table 2 below. Table 2 further includes the COF and RRF data for electrospun tubes of PLGA 82:18 and poly(L-lactic acid) prior to a laser-cutting process. Flow diverter information is provided and is further explained below.

TABLE 2
Chronic outward forces (COF) and radial resistive forces (RRF)

| | Design #5 | Design #3 | Electrospun | Flow | Electrospun |
|---|---|---|---|---|---|
| | (long | (short | tube (PLGA | diverter | tube |
| | struts) | struts) | 82:18) | (Pipeline) | (PLLA) |
| COF | 0.026547 | 0.016698 | 0.298641 | 0.005194 | 2.43 |
| (N/mm) | | | | | |
| RRF | 0.039778 | 0.023012 | 6.72 | 0.012121 | 20.3076 |
| (N/mm) | | | | | |

Stent devices as described herein can be used in any application typical of stents, as would be apparent to one of skill in the art in view of this disclosure. Particularly, stents according to the instant disclosure may include coronary stents, vascular stents, ureteral stents, prostatic stents, esophageal stents, biliary stents and the like.

### Flow Diverter

In one embodiment, the medical device disclosed herein includes a flow diverter. In one embodiment, the flow diverter includes a substantially tubular lattice framework 101 having a plurality of interconnected polymeric electrospun fiber members. The lattice framework 101 may also be made and/or include materials according to embodiments disclosed herein. In another embodiment, the flow diverter may include a substantially tubular lattice framework 101 comprising at least one wire strand formed into a plurality of interconnected members, as disclosed herein. Further, the lattice framework 101 of the flow diverter may include a pattern or design according to embodiments disclosed herein.

The flow diverter may also include a polymeric electrospun mesh 102 contacting at least two of the interconnected members. The lattice framework 101 includes cells or openings between the interconnected members. In one embodiment, the polymeric electrospun mesh 102 surrounds the lattice framework 101 in the form of a covering or wrap to provide a mesh 102 covering the cells of the lattice framework 101. In another embodiment, the polymeric electrospun mesh 102 is positioned between two or more of the polymeric electrospun fiber members to cover the cells of the lattice framework 101. In another embodiment, the polymeric electrospun mesh 102 does not cover or wrap over the lattice framework 101. In some embodiments, the polymeric electrospun mesh 102 is added to the lattice framework 101 after fabricating the lattice framework 101 from a electrospun tube. In one embodiment, the polymeric electrospun mesh 102 is substantially uniform.

The polymeric electrospun mesh 102 further includes a pore size. In one embodiment, the pore size is from about 5 μm to about 500 μm. In another embodiment, the pore size is from about 20 μm to about 400 μm. In one embodiment, the pore size is from about 50 μm to about 300 μm. In yet another embodiment, the pore size is from about 75 μm to about 200 μm. In another embodiment, the pore size is from about 80 μm to about 120 μm. In one embodiment, the pore size is about 100 μm. The pore size may be, for example, about 5 μm, about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, or any range between any two of these values, including endpoints. In some embodiments, the polymeric mesh 102 may be deposited to contact the interconnected members in a manner to control the pore size. In another embodiment, the pores can be created by post-processing, such as a laser-cutting process, to fabricate the pores in the polymeric mesh 102. In one embodiment, the flow diverter is configured to hold liquid while still remaining substantially permeable, to maintain, for example, blood flow through side branches. The flow of liquid through the mesh 102, in some embodiments, may result from increased pressure of the liquid within the flow diverter.

The flow diverter may further include an operational (e.g., expanded) configuration having an operational diameter and operational length, and a delivery (e.g., collapsed) configuration having a delivery diameter and delivery length. The details regarding the operational configuration and delivery configuration, including specific lengths, diameters, and ratios, are discussed above.

In embodiments wherein the substantially tubular lattice framework 101 comprises a plurality of interconnected polymeric electrospun fiber members, the polymer electrospun fiber members may comprise any individual polymer or combination of polymers as disclosed herein. In one embodiment, the interconnected polymeric electrospun fiber members comprise from about 10 wt % to about 30 wt % polyethylene terephthalate and from about 70 wt % to about 90 wt % polyurethane, where the total of polyethylene terephthalate and polyurethane equal 100 wt %. In another embodiment, the interconnected polymeric electrospun fiber members comprise from about 20 wt % polyethylene terephthalate and about 80 wt % polyurethane.

In some embodiments, the interconnected polymeric electrospun fiber members and/or the polymeric mesh 102 may comprise a blend of a first polymer and a second polymer. In some embodiments, the first polymer may be present in an amount of about 10 wt %, about 20 wt %, about 30 wt %, about 40 wt %, about 50 wt %, about 60 wt %, about 70 wt %, about 80 wt %, about 90 wt %, or any range between any two of these values, including endpoints. In some embodiments, the second polymer may be present in an amount of about 10 wt %, about 20 wt %, about 30 wt %, about 40 wt %, about 50 wt %, about 60 wt %, about 70 wt %, about 80 wt %, about 90 wt %, or any range between any two of these values, including endpoints.

In one embodiment, one or more of the interconnected polymeric electrospun fiber members and the polymeric mesh 102 include an additional material. In one embodiment, the additional material is contrast agent, as disclosed herein. In another embodiment, the interconnected polymeric electrospun fiber members include a contrast agent. In one embodiment, the polymeric mesh 102 includes a contrast agent. Any concentration and type of additional material as disclosed herein may be incorporated into the interconnected electrospun fiber members and/or the polymeric mesh 102. In one embodiment, the interconnected polymeric electrospun fiber members include a tantalum. In another embodiment, the tantalum is present in the polymeric electrospun fiber members in amount from about 10 wt % to about 1,500 wt %.

In embodiments wherein the substantially tubular lattice framework 101 comprises a plurality of interconnected polymeric electrospun fiber members, the lattice framework 101 may further comprise a metal core (e.g., DFT wire). In one embodiment, the metal core comprises one or more of stainless steel, gold, titanium, cobalt, chromium, tantalum, nickel, titanium, magnesium, iron, alloys thereof, and combinations thereof. The metal core may include a commercial available metal stent structure. In other embodiments, the metal core material may be laser cut to match a desired pattern for lattice framework 101. The metal material matching the desired lattice pattern may have electrospun fibers deposited on its surface to generate the lattice framework 101 of interconnected electrospun fiber members having a metal core. In another embodiment, the cells or openings between the interconnected electrospun fiber members may be cut out using a laser-cutting process. In one embodiment, the metal core may provide a contrast, such as an X-ray contrast, to the flow diverter. In another embodiment, the metal core may comprise an additional contrast agent, such as gold or one or more gold-based materials.

In one embodiment, the flow diverter has a chronic outward force greater than about 0.0265 N. In another embodiment, the flow diverter has a radial resistive force greater than about 0.039 N. In one embodiment, the flow diverter has a chronic outward force greater than about 0.026 N and a radial resistive force greater than about 0.039 N. Table 1 above includes the COF and RFF for a flow diverter made from a metal core of a nickel/titanium alloy (known as nitinol) electrospun with poly(L-lactic acid). This flow diverter had a COF of 0.0052 N/mm and an RRF of 0.012 N/mm.

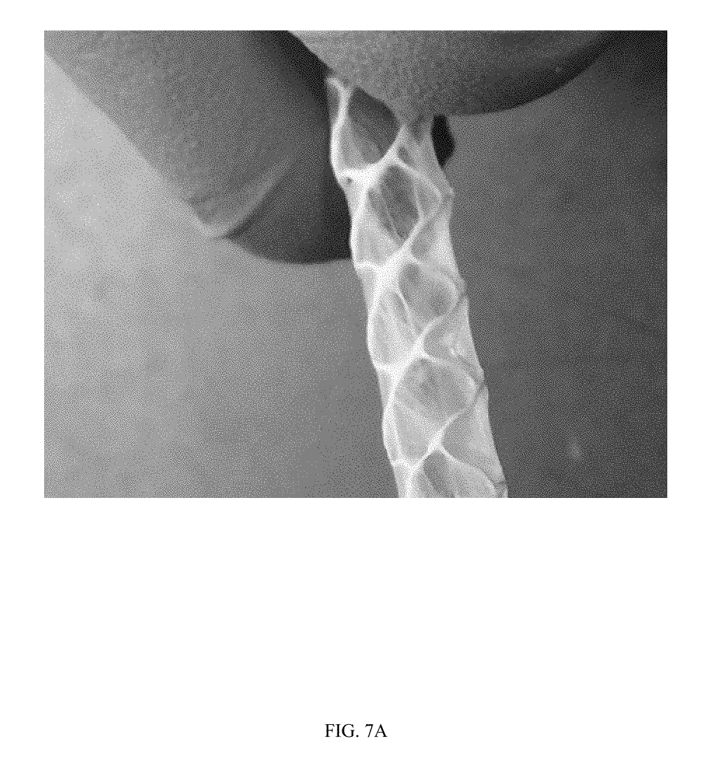

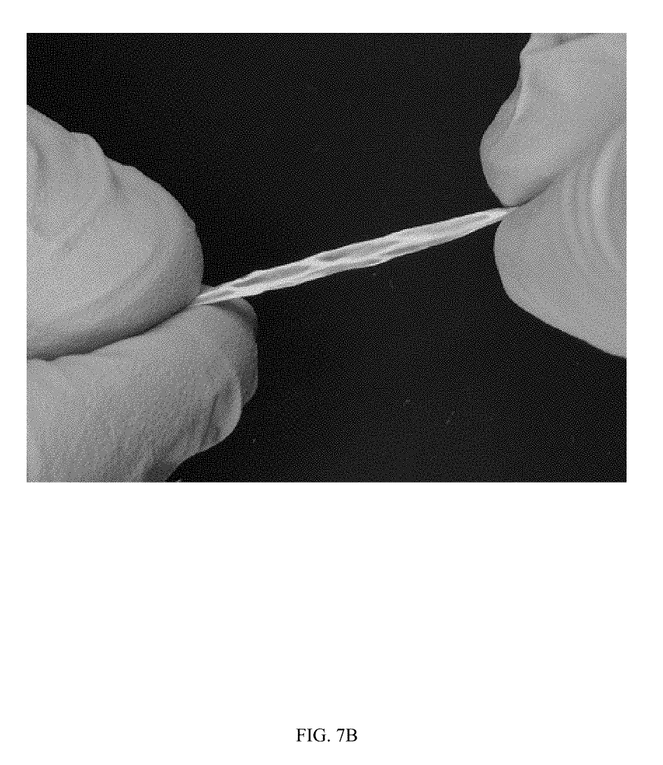

FIG. 7A illustrates a flow diverter having a polymeric mesh 102 coating according to an embodiment. In preparing the flow diverter of FIG. 7A, a polymer solution was prepared by dissolving 8% polyethylene terephthalate (PET) in 1,1,1,3,3,3-hexafluoroisopropanol (wt/wt) under heated conditions of 60° C. The solution was under continuous stirring until the PET was completely dissolved. A 3% polyurethane (PU) solution was made in 1,1,1,3,3,3-hexafluoroisopropanol (wt/wt) with continuous stirring at room temperature until the PU was completely dissolved. The PET and PU solutions were combined to create a final ratio of 20% PET and 80% PU (wt/wt). A portion of the PET/PU solution was initially brushed onto the metal stent structure (e.g., lattice framework or scaffold 101). Adding or applying a portion of the polymer solution to the metal stent structure may improve fiber adhesion of electrospun fibers (e.g., of a polymeric mesh 102) to the metal stent structure as the polymer solution may provide improved tackiness or stickiness for the electrospun fibers to the metal stent structure. The polymer solution was electrospun onto the metal stent structure using 20 gauge blunt-tip needles, a high voltage DC power supply set to +14 kV, and a 15 cm tip-to-substrate distance. When electrospinning the polymer solution onto the metal stent device, the metal stent device can be mounted on the mandrel in multiple configurations. In one configuration, the metal stent device is cantilevered with a negative charge. In another configuration, the metal stent device may be concentrically mounted on the mandrel and electrically isolated from a negatively charged mandrel. In another configuration, the metal stent device may be concentrically mounted on the mandrel and negatively charged. The mandrel may be rotated at various speeds. Further, the mandrel may include a relatively small-diameter mandrel, i.e. about 1 mm diameter up to larger mandrels that are substantially flush with the luminal surface of the metal stent structure. Further, soft mandrels, such as a balloon, or rigid mandrels having cut channels may be employed. Such mandrels may permit the mandrel to be flush with the abluminal surface of the metal stent structure. Various mandrel speeds, sizes and shapes would be apparent to those of skill in the art in view of this disclosure. In some embodiments, a metal stent device and/or a plurality of interconnected electrospun fiber members may be mounted to the mandrel, and a polymer mesh 102 may be electrospun over the stent and/or members, with the mesh 102 contacting two or more lattice components or members, using the electrospinning techniques described herein.

FIG. 7B illustrates the flow diverter of FIG. 7A in a compressed or delivery configuration. The flow diverter may be compressed for delivery of the flow diverter to its intended location. In one embodiment, the pore size of the polymeric electrospun mesh 102 is configured to remain constant as the flow diverter device changes between the delivery length and the operational length. In some embodiments, the density (e.g., porosity or space between fibers) of the polymeric electrospun mesh 102 is configured to remain consistent as the flow diverter device changes between the delivery diameter and the operational diameter. Without wishing to be bound by theory, the pore size and/or density of the polymeric electrospun mesh 102 may remain constant (i.e. the area of the pore size will be substantially maintained) as the device changes between the delivery length and the operational length and/or between the delivery diameter and the operational diameter, by the fibers of the polymeric electrospun mesh 102 translating, moving, or sliding past one another, thereby changing the shape of the pores, but not their area. Flow diverters described herein may have less foreshortening during delivery and operation over currently existing flow diverters, resulting in improved ease of use and deployment. The compressed or delivery configuration allows for delivery of the flow diverter via a microcatheter, as disclosed herein. Other delivery methods are also contemplated, and such methods would be apparent to one of skill in the art in view of this disclosure.

The flow diverters described herein may be used to treat an aneurysm. In one embodiment, the aneurysm includes a cerebral aneurysm. The purpose of a flow diverter is to divert blood flow away from the weakened area, i.e., aneurysm, wherein the flow diverter is placed in an artery leading to the aneurysm, while still allowing appropriate flow to any vessels adjacent to the aneurysm. Flow diverters described herein may be used to treat any condition as would be apparent to one of skill in the art in view of this disclosure. Flow diverters according to the instant disclosure create an impedance by the electrospun polymeric mesh 102. This impedance results in a reduction in (but not necessarily elimination of) blood flow into the aneurysm, causing a pressure imbalance within the aneurysm that leads to an inflammatory response. The inflammatory response may be followed by a healing of an aneurysm. The electrospun polymeric mesh 102 and lattice framework 101 of the instant flow diverters provide biocompatibility and biomimicry that may accelerate the rate of cell adhesion and endothelialization to close off the aneurysm from the parent artery, and remodel the artery wall to ultimately close off the aneurysm.

The instant disclosure is also directed to methods of manufacturing a medical device, employing the electrospinning techniques described herein. In one embodiment, the medical device comprises a stent. In another embodiment, the medical device comprises a flow diverter. The method includes providing a mandrel and a polymer injection system for electrospinning the polymer solution onto the mandrel. The polymer injection system may be positioned at a distance from the mandrel. In one embodiment, the distance between the mandrel and the polymer injection system is about 20 cm. A charge may be applied to one or more of the mandrel and the polymer injection system. In one embodiment, applying a charge comprises applying a +11.1 kV charge to the mandrel and a −4.0 kV charge to the metal lattice framework 101 on the mandrel. The polymer injection system is loaded with a polymer solution. Suitable polymer solutions are discussed herein. The mandrel is spun at a rotation speed. In one embodiment, the mandrel is rotated at about 6,500 rpm. While the mandrel is rotating, the polymer solution is ejected from the polymer injection system at a flow rate onto the mandrel to generate a polymer stream for an electrospun fiber. In one embodiment, the flow rate is about 5 mL/hour.

In some embodiments, the mandrel may include a metal material where the polymer solution is ejected onto the metal material. In one embodiment, the metal material is a metal stent structure. In another embodiment, the metal material includes a lattice framework 101. The polymer solution is ejected onto the mandrel to form a tubular section. The tubular section is removed from the mandrel and processed. In some embodiments, a first portion of the polymer solution is applied to the metal lattice framework 101 prior to placing the metal lattice framework 101 on the mandrel. In one embodiment, the tubular section may be processed by dipping the tubular section in a solvent. In another embodiment, the solvent comprises IPA.

In one embodiment, the tubular section may be processed by laser cutting the tubular section to form a lattice framework 101 having a plurality of interconnected polymeric electrospun fiber members. In one embodiment, the lattice framework 101 has a pattern. Suitable patterns for the lattice framework 101 are disclosed herein. In one embodiment, a polymeric electrospun mesh 102 is applied to the lattice framework 101. The polymeric electrospun mesh 102 may contact at least two of the interconnected polymeric electrospun fiber members. The polymeric electrospun mesh 102 further includes a pore size. As noted, the lattice framework 101 includes cells or openings between adjacent portions of the interconnected polymeric electrospun fibers. The polymeric electrospun mesh 102 may contact the interconnected polymeric electrospun fiber members to substantially cover these cells or openings. In some embodiments, the pores of the polymeric electrospun fiber mesh 102 are created by laser-cutting.

In some embodiments, the lattice framework or scaffold 101 is constructed of interconnected or braided metal members, wires, or strands (e.g., DFT wiring, nitinol). The framework may have a suitable pattern as described above. The polymeric mesh 102 may be applied (e.g., directly) to the metal lattice framework 101 to cover the framework (e.g., completely or partially). The polymeric mesh 102 may be constructed from a plurality of interconnected polymeric electrospun nanofibers. For example, the mesh 102 may extend around a body of the framework and not the ends 103. The mesh 102 may cover the framework to prevent exposure of the metal material to blood flow through a vessel or the flow diverter. The mesh 102 may also accelerate occlusion and promote endothelial response as described herein (e.g., include a porous architecture mimicking an ECM matrix of tissue surrounding an implant site of the device). Further, polymer solution may be applied to the lattice framework 101 prior to applying the mesh 102 onto the framework.

The polymeric electrospun mesh 102 may contact at least two of the interconnected members of the metallic lattice framework 101. The polymeric electrospun mesh 102 further includes a pore size. As noted, the lattice framework 101 includes openings between adjacent portions of the interconnected members. The polymeric electrospun mesh 102 may contact the interconnected members to substantially cover these cells or openings. In some embodiments, the pores of the polymeric electrospun fiber mesh 102 are created by laser-cutting.

### Occlusion Device

In some embodiments, a medical device may comprise a lattice framework 101 (e.g., made of DFT wiring, nitinol or other suitable metallic material) including at least one strand formed into a plurality of interconnected members, as described herein. The medical device may also comprise a polymeric electrospun mesh 102 contacting at least two of the interconnected members, as described herein. In some embodiments, such a polymeric electrospun mesh 102 may accelerate or improve a rate of occlusion to prevent or stop fluid (e.g., blood) flow past or through the medical device. The polymeric electrospun mesh 102 may also have a pore size, as described herein. Such a medical device may be manufactured or treated by any of the methods or processes described herein. Further, the lattice framework 101 may also include both a metallic material and interconnected nanofiber members. In some embodiments, a resorbable layer of nanofiber members or mesh 102 extends or is positioned on the polymeric mesh 102.

In some embodiments, the at least one strand may comprise a wire, as described herein. In other embodiments, the at least one strand may comprise at least one polymeric electrospun fiber, as described herein. In certain embodiments, the at least one strand may comprise a combination of wire and at least one polymeric electrospun fiber. In some embodiments, the medical device may further comprise an adhesive between the lattice framework 101 and the polymeric electrospun mesh 102. In some embodiments, the adhesive may be configured to bond the lattice framework 101 to the polymeric electrospun mesh 102. The adhesive may comprise, for example, cyanoacrylate, silicone, or a dilute polymer solution such as, for example, 1wt % PU dissolved in HFIP.

In some embodiments, such a medical device may have an operational configuration (e.g., expanded) and a delivery configuration (e.g., collapsed) as described herein. In certain embodiments, the operational configuration may comprise an operational diameter and an operational length as described herein, and the delivery configuration may comprise a delivery diameter and a delivery length as described herein. In certain embodiments, the operational diameter may be greater than the delivery diameter, and the operational length may be less than the delivery length, as described herein. In some embodiments, for example, diameter of the occlusion device may range from about 1 mm to about 20 mm (e.g., between delivery and operational configurations). In some embodiments, length of the occlusion device may range from about 3 mm to about 100 mm (e.g., between operational and delivery configurations).

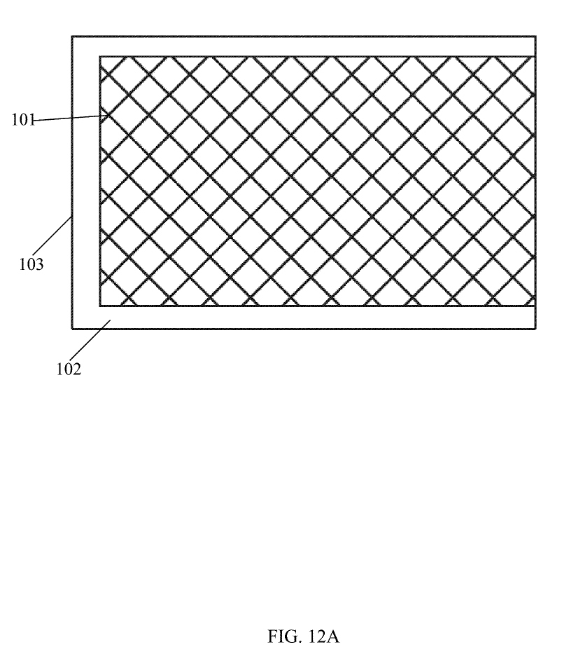

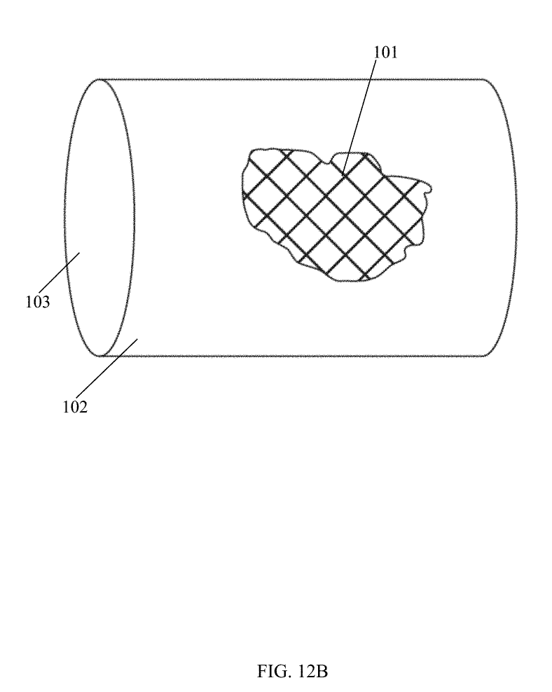

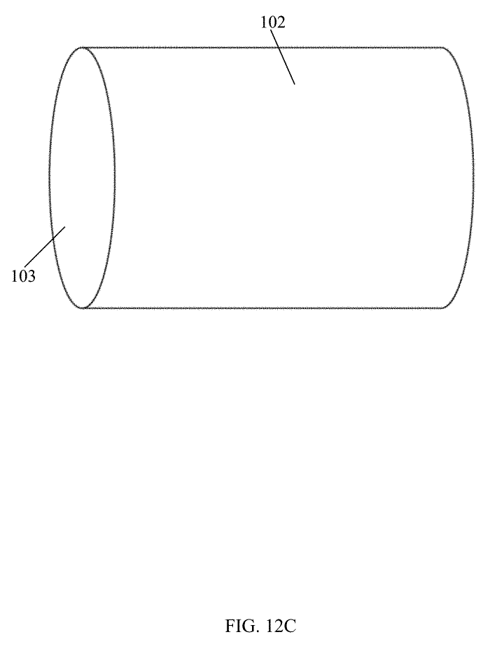

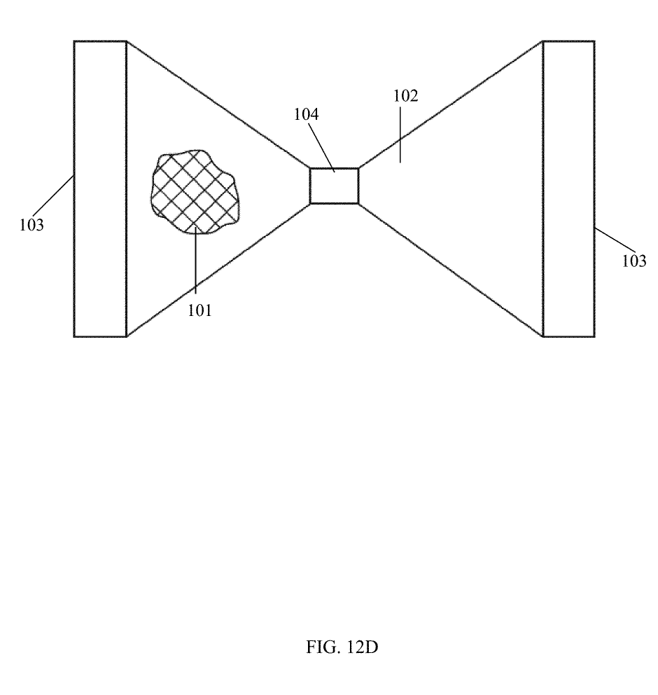

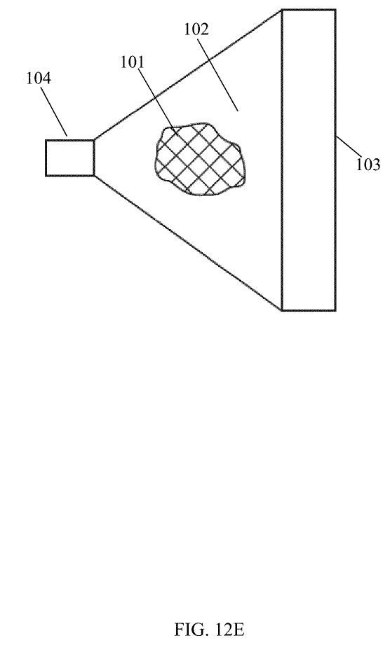

In some embodiments, the polymeric electrospun mesh 102 may cover, either fully or partially, one or both ends 103 of the medical device. In certain embodiments, the polymeric electrospun mesh 102 may extend across or cover a body (e.g., scaffold or lattice framework 101) of the medical device (e.g., between the ends 103 of the medical device). In other embodiments, the mesh 102 extends partially across or only covers partially the body of the medical device. In still further embodiments, the mesh 102 only extends or covers, either fully or partially, one or both ends 103 of the medical device. The mesh 102 may prevent exposure of the metallic portions of the framework or scaffold 101 from blood flow. Schematic illustrations of such a device are shown in FIG. 12A, FIG. 12B, and FIG. 12C. In certain embodiments, the polymeric electrospun mesh 102 may tightly cover one or both ends 103 of the medical device, much like an end of a drum.

In some embodiments, the lattice framework 101 may be substantially tubular or cylindrical (FIGS. 12A-12C), as described herein. In other embodiments, the lattice framework 101 may have a “bowtie” shape, as illustrated in FIG. 12D. A “bowtie” shaped configuration may provide additional multi-directional anchoring of the medical device (e.g., to a vessel or artery wall). In still other embodiments, the lattice framework 101 may have a cone or cone-like shape, as illustrated in FIG. 12E. In certain embodiments, the medical device may further comprise a marker 104, as illustrated in FIG. 12D and FIG. 12E. In some embodiments, the marker 104 may, for example, comprise a metal, contrast agent, or other radiopaque material. In some embodiments, the marker 104 may include a metal such as, for example, gold, platinum, tantalum, or tungsten.

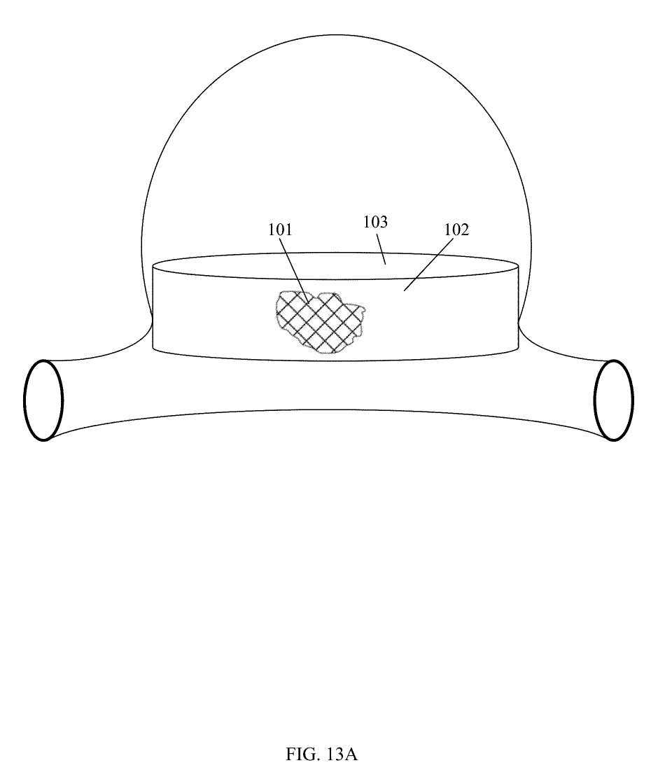

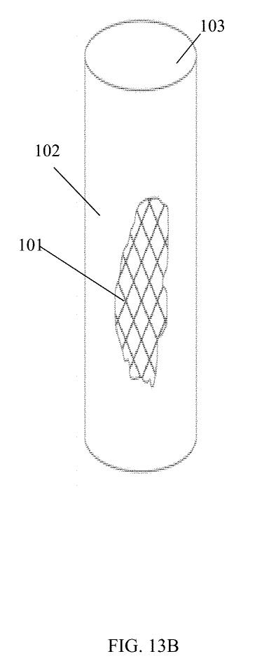

In certain embodiments, such a medical device may be, for example, a vascular plug, used to occlude a vessel, either partially or fully. In other embodiments, such a medical device may be used “intrasaccularly” within the sac of an aneurysm to treat or occlude it. See FIG. 13A. Without wishing to be bound by theory, a medical device as described herein may help occlude a vessel by blocking at least a portion of blood flow while simultaneously encouraging endothelialization to occur. When blood becomes stagnant, it clots. Such a medical device may help to occlude a sac of an aneurysm by a similar method.

For example, intrasaccular devices, as described herein, may be positioned in an expanded configuration within the blood vessel and conform to a body and/or neck portion of an aneurysm to substantially occlude blood or other fluid flow into the aneurysm as illustrated in FIG. 13A. FIG. 13B shows the intrasaccular device in a compressed configuration for delivery. Intrasaccular devices having a coil-like structure are described in, for example, U.S. Pat. Pub. No. 2017/0071607, which is incorporated herein by reference in its entirety.

In some embodiments, an intrasaccular device may include a scaffold or lattice framework 101 having a metal core (e.g., DFT wire) and interconnected polymeric electrospun fiber members deposited on the metal core as described herein. In some embodiments, the framework, fiber members, and/or mesh 102 includes a hydrophilic component (e.g., for improved deployability). The device may include a polymeric electrospun mesh 102 contacting or covering the electrospun fiber members (e.g., cells or openings). In some embodiments, the polymeric mesh 102 contacts or covers a metallic lattice framework 101 without an intermediate layer of interconnected nanofiber members as described above. The polymeric mesh 102 may be applied directly to the metallic framework or after a polymer solution is applied to the framework as described herein. The mesh 102 may extend across or cover partially and/or completely: one end 103, two ends 103, and/or the body (e.g. lattice framework or scaffold 101) of the device as described above. The mesh 102 may include a plurality of interconnected fiber members.

The mesh 102 may have a porous architecture. Such an architecture may mimic the extracellular matrix (ECM) of the tissue surrounding the placement location (e.g., body and/or neck of an aneurysm) of the instrasaccular device. The mimicking of the ECM is believed to permit cells to grow on the ECM and induce tissue regeneration leading to a rapid re-endothelialization at the interface between the device and the tissue surrounding the placement location. In another embodiment, the polymeric electrospun fiber members which form the lattice framework 101 may alternatively (or in addition to the mesh 102) also have a fibrous architecture. The metal core may provide support for anchoring the polymer mesh 102 within the aneurysm. Providing a polymeric electrospun mesh 102 may improve endothelial response (e.g., promote endothelial growth) as described herein, increase or expedite occlusion time, reduce amount of metal required, and/or improve flexibility for positioning the device (e.g., of occlusion or intrasaccular device). An expandable and/or flexible intrasaccular or occlusion device as described herein may more readily conform to an aneurysm or other vessel relative to traditional devices that are typically more rigid or have predetermined shaped that may prevent complete or substantial occlusion. For example, some traditional devices may have a predetermined shape that leaves openings at a neck portion of an aneurysm. Devices as described herein may provide more complete occlusions or sealing (e.g., at a neck of an aneurysm).

The intrasaccular device may have an operational diameter greater than a delivery diameter, and an operational length less than a delivery length, as described herein. In some embodiments, diameter of the intrasaccular device may range from about 1 mm to about 35 mm (e.g., between delivery and operational diameters). In some embodiments, length of the occlusion device may range from about 1.5 mm to about 50 mm (e.g., between operational and delivery lengths).

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1.-65. (canceled)

66. An implantable medical device comprising:
a lattice framework having a metal core and a plurality of interconnected polymeric electrospun fiber members deposited on the metal core; and
a polymeric electrospun mesh contacting at least two of the plurality of interconnected polymeric electrospun fiber members and having a pore size,
wherein the medical device has an expanded configuration comprising an expanded diameter and a length, and a collapsed configuration comprising a collapsed diameter and a length, and wherein the expanded diameter is greater than the collapsed diameter.

67. The implantable medical device of claim 66, wherein the length in the expanded configuration is less than or equal to the length in the collapsed configuration.

68. The implantable medical device of claim 66, wherein the polymeric electrospun mesh 102 contacts at least two adjacent interconnected polymeric electrospun fiber members.

69. The implantable medical device of claim 66, wherein the metal core comprises a drawn filled tubing wire.

70. The implantable medical device of claim 66, wherein the polymeric electrospun mesh 102 comprises a blend of at least two polymers.

71. The implantable medical device of claim 66, wherein the polymeric electrospun fiber members comprise a blend of at least two polymers.

72. The implantable medical device of claim 66, wherein the polymeric electrospun mesh 102 covers at least one end of the medical device and is configured to occlude a blood vessel.

73. The implantable medical device of claim 72, wherein the polymeric electrospun mesh 102 extends across the lattice framework covering openings between the plurality of polymeric electrospun fiber members.

74. The implantable medical device of claim 73, wherein the polymeric electrospun mesh 102 covers a second end of the medical device.

75. The implantable medical device of claim 66, wherein the lattice framework comprises a repeating pattern in a shape of: bricks, hexagons, fish scales, vertical circles, horizontal circles, vertical diamonds, horizontal diamonds, vertical zig-zags, horizontal zig-zags, vertical sinusoids, or horizontal sinusoids.

76. The implantable medical device of claim 66, wherein at least one of the polymeric electrospun fiber members, the polymeric electrospun mesh, or the metal core comprises a contrast agent.

77. The implantable medical device of claim 66, wherein the lattice framework comprises a substantially tubular shaped, a substantially cone shaped, or a substantially bow-tie shaped configuration and is configured to occlude a blood vessel.

78. The implantable medical device of claim 66, wherein the pore size of the polymeric electrospun mesh s from about 5 μm to about 500 μm.

79. The implantable medical device of claim 66, wherein the pore size of the polymeric electrospun mesh is configured to remain constant as the implantable medical device changes between the expanded diameter and the collapsed diameter.

80. The implantable medical device of claim 66, wherein a density of the polymeric electrospun mesh is configured to remain constant as the implantable medical device changes between the expanded diameter and the collapsed diameter.

81. The implantable medical device of claim 66, wherein a polymer solution is added to the metal core prior to depositing the polymeric electrospun fibers onto the metal core.

82. The implantable medical device of claim 66, wherein the plurality of interconnected polymeric electrospun fiber members comprise a porous architecture mimicking an extracellular matrix of tissue surrounding an implant site.

83. The implantable medical device of claim 66, wherein the lattice framework is configured to divert fluid flow through a blood vessel away from a patient aneurysm.

84. The implantable medical device of claim 66, wherein a pore size of the mesh is configured to prevent fluid flow therethrough.

85. An embolization device configured to be inserted into and conform to a shape of an aneurysm of a patient, the embolization device comprising:
a coil having a substantially tubular metal core and a plurality of interconnected polymeric electrospun fiber members deposited on the substantially tubular metal core, the metal core comprising a drawn filled tubing wire, and a hydrophilic component.

86. The embolization device of claim 85, wherein the embolization device has an expanded configuration comprising an expanded diameter and an expanded length, and a collapsed configuration comprising a collapsed diameter and a collapsed length, and wherein the expanded diameter is greater than the collapsed diameter.

87. The embolization device of claim 85, further comprising a polymeric electrospun mesh 102 contacting at least two of the plurality of interconnected polymeric electrospun fiber members and having a pore size.

88. The embolization device of claim 85, wherein at least one of the polymeric electrospun fiber members or the metal core comprises a contrast agent.

89. The embolization device of claim 85, wherein a polymer solution is added to the metal core prior to depositing the polymeric electrospun fibers onto the metal core.

90. The embolization device of claim 85, wherein the plurality of interconnected polymeric electrospun fiber members comprise a porous architecture mimicking an extracellular matrix of tissue surrounding an implant site.

91. A method of manufacturing an implantable medical device, the method comprising:
applying a charge to at least one of a mandrel or a polymer injection system, the polymer injection system spaced apart from the mandrel at a distance;
loading the polymer injection system with a polymer solution;
providing a metal core material on the mandrel;
spinning the mandrel at a rotation speed;
ejecting the polymer solution at a flow rate to deposit polymeric electrospun fiber members onto the metal core material on the mandrel;
removing the metal core material with the deposited polymeric electrospun fiber members from the mandrel; and
processing the removed metal core material with the deposited polymeric electrospun fiber members.

92. The method of claim 91, wherein the metal core material comprises a lattice framework on the mandrel.

93. The method of claim 91, wherein the processing comprises laser cutting the removed metal core material with the deposited polymeric electrospun fiber members to form a lattice framework having a plurality of interconnected polymeric electrospun fiber members.

94. The method of claim 91, further comprising contacting a polymeric electrospun mesh to at least two adjacent polymeric electrospun fiber members to substantially cover openings between the adjacent polymeric electrospun fibers, the polymeric electrospun mesh having a pore size.

95. The method of claim 91, wherein the metal material comprises a drawn filled tubing wiring.

96. The method of claim 91, wherein a portion of the polymer solution is applied to the metal material prior to ejecting the polymer solution at a flow rate to deposit the polymeric electrospun fiber members onto the metal material.

97. The method of claim 91, wherein the processing comprises at least one of: a dip-coating treatment, a heat treatment, or a solvent treatment.

98. The method of claim 91, wherein the medical device is one of: a flow diverter, a vascular plug, or an embolization coil.
